(19) United States
(12) Patent Application Publication
(10) Pub. No.: US 2024/0409559 A1
(43) Pub. Date: Dec. 12, 2024

(54) CRYSTALLINE FORM OF AZALACTAM COMPOUND

(71) Applicant: PFIZER INC., NEW YORK, NY (US)

(72) Inventors: Fengjuan CAO, San Diego, CA (US); Martha Alicia ORNELAS, San Diego, CA (US)

(73) Assignee: PFIZER INC., NEW YORK, NY (US)

(21) Appl. No.: 18/698,272

(22) PCT Filed: Oct. 4, 2022
(86) PCT No.: PCT/IB2022/059439

§ 371 Date: Apr. 3, 2024

Related U.S. Application Data
(60) Provisional application No. 63/252,457, filed on Oct. 5, 2021.
(60) Provisional application No. 63/273,482, filed on Oct. 29, 2021.

(51) Int. Cl. C07D 519/00; A61K 31/444

(52) U.S. Cl. CPC C07D 519/00; A61K 31/444

(57) ABSTRACT

This disclosure relates to a crystalline form of 4-[(1R)-1-aminopropyl]-2-{6-[(5S)-5-methyl-6,7-dihydro-5H-pyrro-lo[2,1-c][1,2,4]triazol-3-yl]pyridin-2-yl}-6-[(2R)-2-methylpyrrolidin-1-yl]-2,3-dihydro-1H-pyrrolo[3,4-c]pyridin- 1-one (PF-07265028) free base and method of making thereof. The disclosure also relates to pharmaceutical compositions comprising this crystalline form, and to methods of using the crystalline form and such compositions for the treatment of abnormal cell growth, such as cancer, in a mammal.

### BACKGROUND

This disclosure relates to a crystalline form of 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one, having the structure of

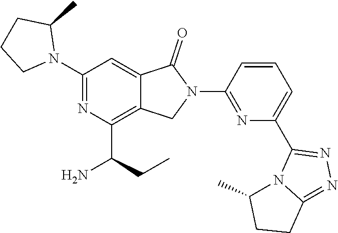 [SMILES: CC[C@@H](N)c1nc(N2CCC[C@H]2C)cc2c1CN(c1cccc(-c3nnc4n3[C@@H](C)CC4)n1)C2=O]

(also referred to herein as PF-07265028) free base (Form 2), to pharmaceutical compositions comprising Form 2, and to methods of using Form 2 and such compositions in the treatment of abnormal cell growth, such as cancer, in mammals.

Preparation of PF-07265028, isolated either as an amorphous form or a mixture of an amorphous form and a crystalline hydrate form (Form 1), is disclosed in International Patent Publication No. WO2021/220185, the contents of which are incorporated herein by reference in their entirety.

The present disclosure provides crystalline form of anhydrous PF-07265028 having desirable properties, such as high crystallinity, high purity, low hygroscopicity, favorable dissolution or mechanical properties, improved manufacturability or filterability, and/or favorable stability.

### BRIEF SUMMARY

The present disclosure provides a crystalline form of PF-07265028 free base (Form 2). The disclosure also provides pharmaceutical compositions, methods of preparing and methods of using PF-07265028 free base (Form 2) for treating diseases and conditions which are affected by HPK1 inhibition.

Form 2 of PF-07265028 free base is characterized by one or more of the following methods: (1) powder X-ray diffraction (PXRD) (2θ); (2) Raman spectroscopy (cm⁻¹); or (3) ¹³C solid state NMR spectroscopy (ppm).

In a first aspect, the disclosure provides PF-07265028 free base (Form 2), which is characterized by having:
(1) a powder X-ray diffraction (PXRD) pattern (2θ) comprising: (a) one, two, three, four, five or more than five peaks selected from the group consisting of the peaks in Table 2 in 2θ±0.2°; or (b) peaks at 2θ values essentially the same as shown in FIG. 1; or
(2) a Raman spectrum comprising: (a) one, two, three, four, five, or more than five wavenumber (cm⁻¹) values selected from the group consisting of the values in Table 3 in cm⁻¹±3 cm⁻¹; or (b) wavenumber (cm⁻¹) values essentially the same as shown in FIG. 2; or
(3) a ¹³C solid state NMR spectrum (ppm) comprising: (a) one, two, three, four, five, or more than five resonance (ppm) values selected from the group consisting of the values in Table 4 in ppm±0.2 ppm; or (b) resonance (ppm) values essentially the same as shown in FIG. 3; or
a combination of any two or three of the foregoing embodiments (1)(a)-(c), (2)(a)-(c), and (3)(a)-(c), provided they are not inconsistent with each other.

In another aspect, the disclosure provides PF-07265028 free base (Form 2), which is characterized by having:
(1) a powder X-ray diffraction (PXRD) pattern (2θ) comprising: (a) three, four, five or more than five peaks selected from the group consisting of the peaks in Table 2 in 2θ±0.2°; or (b) peaks at 2θ values essentially the same as shown in FIG. 1; or
(2) a Raman spectrum comprising: (a) three, four, five, or more than five wavenumber (cm⁻¹) values selected from the group consisting of the values in Table 3 in cm⁻¹±3 cm⁻¹; or (b) wavenumber (cm⁻¹) values essentially the same as shown in FIG. 2; or
(3) a ¹³C solid state NMR spectrum (ppm) comprising: (a) three, four, five, or more than five resonance (ppm) values selected from the group consisting of the values in Table 4 in ppm±0.2 ppm; or (b) resonance (ppm) values essentially the same as shown in FIG. 3; or
a combination of any two or three of the foregoing embodiments (1)(a)-(c), (2)(a)-(c), and (3)(a)-(c), provided they are not inconsistent with each other.

In another aspect, the disclosure provides a method of preparing a crystalline form of PF-07265028 (Form 2) free base.

In another aspect, the disclosure further provides a pharmaceutical composition comprising PF-07265028 free base (Form 2), according to any of the embodiments described herein, and a pharmaceutically acceptable carrier or excipient.

In another aspect, the disclosure provides a method of treating abnormal cell growth in a mammal, including a human, comprising administering to the mammal a therapeutically effective amount of PF-07265028 free base (Form 2).

In another aspect, the disclosure provides a method of treating abnormal cell growth in a mammal, comprising administering to the mammal a therapeutically effective amount of a pharmaceutical composition comprising PF-07265028 free base (Form 2), according to any of the aspects or embodiments described herein.

In another aspect, the disclosure provides use of PF-07265028 free base (Form 2), or a pharmaceutical composition comprising the PF-07265028 free base (Form 2), according to any of the aspects or embodiments described herein, in a method of treating abnormal cell growth in a mammal.

In another aspect, the disclosure provides PF-07265028 free base (Form 2), or a pharmaceutical composition comprising the PF-07265028 free base (Form 2), according to any of the aspects or embodiments described herein, for use in a method of treating abnormal cell growth in a mammal.

In yet another aspect, the disclosure provides use of PF-07265028 free base (Form 2), according to any of the aspects or embodiments described herein, in the manufacture of a medicament for the treatment of abnormal cell growth in a mammal.

In frequent embodiments, the abnormal cell growth is cancer. In one embodiment, anti-tumor immunity is limited by HPK1.

In some embodiments, the abnormal cell growth is cancer, wherein the cancer is selected from the group consisting of brain cancer, head/neck cancer (including squamous cell carcinoma of the head and neck (SCCHN)), prostate cancer, bladder cancer (including urothelial carcinoma, also known as transitional cell carcinoma (TCC)), lung cancer (including squamous cell carcinoma, small cell lung cancer (SCLC), and non-small cell lung cancer (NSCLC)), breast cancer, ovarian cancer, bone cancer, colorectal cancer, kidney cancer, liver cancer (including hepatocellular carcinoma (HCC)), pancreatic cancer, esophageal cancer (including squamous cell carcinoma (SCCGC)), gastric cancer, gastroesophageal junction cancer, thyroid cancer, cervical cancer, uterine cancer, and/or renal cancer.

In some embodiments, the cancer is head and neck squamous cell carcinoma, urothelial carcinoma, non-small cell lung cancer, gastric cancer, and/or gastroesophageal junction cancer.

### BRIEF DESCRIPTION OF THE DRAWINGS

FIG. 1 shows PXRD pattern of PF-07265028 free base (Form 2).

FIG. 2 shows FT-Raman spectrum of PF-07265028 free base (Form 2).

FIG. 3 shows Carbon CP MAS spectrum of PF-07265028 free base (Form 2).

FIG. 4 shows PXRD pattern of PF-07265028 (Form 1).

FIG. 5 shows PXRD pattern of PF-07265028 (Form 1 (Pattern D)).

FIG. 6 shows PXRD pattern of PF-07265028 (Form 1 (Pattern V)).

FIG. 7 shows PXRD pattern of PF-07265028 (Form 1 (Pattern AB)).

FIG. 8 shows PXRD pattern of PF-07265028 (Form 1 (Pattern E)).

### DETAILED DESCRIPTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.
E1. A crystalline form of 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one (PF-07265028) free base (Form 2), having a powder X-ray diffraction (PXRD) pattern measured using a copper radiation source comprising peaks at 2θ values of: 9.1, 11.6, and 18.5°±0.2°.
E2. The crystalline form of embodiment E1, having a PXRD pattern further comprising a peak at the 2θ value of: 16.7°±0.2°.
E3. The crystalline form of embodiment E1 or E2, having a Raman spectrum comprising one or more wavenumber (cm⁻¹) values selected from the group consisting of: 1700, 1402, and 2827 cm⁻¹±3 cm⁻¹.
E4. The crystalline form of any one of embodiments E1 to E3, having a ¹³C solid state NMR spectrum comprising one or more resonance (ppm) values selected from the group consisting of: 159.5, 145.8 and 152.6 ppm±0.2 ppm relative to an external standard of L-alanine, setting its upfield resonance to 177.8 ppm.
E5. A crystalline form of PF-07265028 free base (Form 2), having a ¹³C solid state NMR spectrum comprising resonance (ppm) values of: 159.5, 145.8 and 152.6 ppm±0.2 ppm, relative to an external standard of L-alanine, setting its upfield resonance to 177.8 ppm.
E6. The crystalline form of embodiment E5, having a ¹³C solid state NMR spectrum further comprising resonance (ppm) values of 119.8 ppm±0.2 ppm.
E7. The crystalline form of embodiment E5 or E6, having a ¹³C solid state NMR spectrum further comprising resonance (ppm) values of 168.1 ppm±0.2 ppm.
E8. The crystalline form of any one of embodiments E5 to E7, having a Raman spectrum comprising one or more wavenumber (cm⁻¹) values selected from the group consisting of: 1700, 1402, and 2827 cm⁻¹±3 cm⁻¹.
E9. A crystalline form of PF-07265028 free base (Form 2), having a Raman spectrum comprising wavenumber (cm⁻¹) values of: 1700, 1402, and 2827 cm¹±3 cm⁻¹.
E10. The crystalline form of embodiment E9, having a Raman spectrum further comprising a wavenumber value of 1441 cm⁻¹±3 cm⁻¹.
E11. The crystalline form of embodiment E9 or E10, having a Raman spectrum further comprising a wavenumber value of 2996 cm⁻¹±3 cm⁻¹.
E12. A crystalline form of PF-07265028 free base (Form 2), having: (a) a powder X-ray diffraction (PXRD) pattern measured using a copper radiation source comprising peaks at 2θ values of: 9.1, 11.6, and 18.5°±0.2°; (b) a Raman spectrum comprising one or more wavenumber (cm⁻¹) values selected from the group consisting of: 1700, 1402, and 2827 cm⁻¹±3 cm⁻¹; or (c) a ¹³C solid state NMR spectrum comprising one or more resonance (ppm) values selected from the group consisting of: 159.5, 145.8 and 152.6 ppm±0.2 ppm, relative to an external standard of L-alanine, setting its upfield resonance to 177.8 ppm ppm; or a combination of two or more of (a), (b), and (c).
E13. The crystalline form of any one of embodiments E1 to E12 which is substantially pure.
E14. The crystalline form of any one of embodiments E1 to E13, wherein the crystalline form is anhydrous.
E15. A pharmaceutical composition comprising the crystalline form of PF-07265028 free base according to any one of embodiments E1 to E14, and a pharmaceutically acceptable carrier or excipient.
E16. A method of treating abnormal cell growth in a mammal comprising administering to the mammal a therapeutically effective amount of the crystalline form of PF-07265028 free base according to any one of embodiments E1 to E14, or a pharmaceutical composition according to embodiment E15.
E17. The method of embodiment E16, wherein the abnormal cell growth is cancer.
E18. The method of embodiment E17, wherein the cancer is selected from the group consisting of brain cancer, head and neck cancer, prostate cancer, bladder cancer, lung cancer, breast cancer, ovarian cancer, bone cancer, colorectal cancer, kidney cancer, liver cancer, pancreatic cancer, esophageal cancer, gastric cancer, gastroesophageal junction cancer, thyroid cancer, cervical cancer, uterine cancer, and renal cancer.
E19. The method of any one of embodiments E16 to E18, wherein anti-tumor immunity is limited by HPK1.
E20. Use of a crystalline form of PF-07265028 free base according to any one of embodiments E1 to E14 in a method of treating abnormal cell growth in a mammal.
E21. The use of embodiment E20, wherein the abnormal cell growth is cancer.
E22. The crystalline form of PF-07265028 free base according to any one of embodiments E1 to E14 for use in a method of treating abnormal cell growth in a mammal.
E23. The crystalline form for use of embodiment E22, wherein the abnormal cell growth is cancer.
E24. A method of preparing a crystalline form of 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one (PF-07265028) free base (Form 2) according to any one of embodiments E1 to E14, the method comprising steps of:
   providing PF-07265028 free base in a first solvent to form a first solvent solution;
   adding the first solvent solution to a second solvent at a temperature from about 73° C. to about 90° C. to form a first-second-solvent solution;
   removing proportion of the solvent from the first-second-solvent solution while maintaining a ratio (v/v) of the first solvent to the second solvent in the range from about 20:80 to about 2:98 to obtain a slurry; and
   cooling the slurry to obtain the crystalline form of PF-07265028 free base (Form 2).
E25. The method of embodiment E24, wherein the first solvent is selected from the group consisting of dichloromethane, ethyl ether, acetone, and mixtures thereof.
E26. The method of embodiment E24 or E25, wherein the second solvent is selected from the group consisting of isopropyl acetate n-propyl acetate, isobutyl acetate, t-butyl acetate, and mixtures thereof.

### Definitions

As used herein, the singular form “a”, “an”, and “the” include plural references unless indicated otherwise. For example, “a” substituent includes one or more substituents.

The term “about” means having a value falling within an accepted standard of error of the mean, when considered by one of ordinary skill in the art. The term “about” when used to modify a numerically defined parameter means that the parameter may vary by as much as 10% above or below the stated numerical value for that parameter.

As used herein, the term “essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (2θ) will show some variability, typically as much as ±0.2°. Further, one skilled in the art will appreciate that relative peak intensities will show inter-apparatus variability, as well as variability due to the degree of crystallinity, preferred orientation, prepared sample surface, and other factors known to those skilled in the art and should be taken as qualitative measures only. Similarly, Raman spectrum wavenumber (cm⁻¹) values show variability, typically as much as ±3 cm⁻¹, while ¹³C solid state NMR spectrum (ppm) show variability, typically as much as ±0.2 ppm.

The term “crystalline” as used herein, means having a regularly repeating arrangement of molecules or external face planes. Crystalline forms may differ with respect to thermodynamic stability, physical parameters, x-ray structure and preparation processes.

The term “anhydrous” as used herein, refers to a crystalline form that does not contain water molecules crystalline lattice.

The term “therapeutically effective amount” as used herein refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.

As used herein, “mammal” refers to a human or animal subject. In certain embodiments, the mammal is a human.

The term “treating”, as used herein, unless otherwise indicated, means reversing, alleviating, inhibiting the progress of, or delaying the recurrence of the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous). In frequent embodiments of the methods provided herein, the abnormal cell growth is cancer.

As used herein “cancer” refers to any malignant and/or invasive growth or tumor caused by abnormal cell growth. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one.

As used herein, the term “substantially pure” means that the crystalline form contains at least 90%, preferably at least 95%, more preferably at least 97%, and most preferably at least 99% by weight of the indicated crystalline form (e.g., Form 2). Alternatively, it will be understood that “substantially pure” means that the crystalline form contains less than 10%, preferably less than 5%, more preferably less than 3%, and most preferably less than 1% by weight of impurities, including other polymorphic, solvated or amorphous forms.

The disclosure described herein may be suitably practiced in the absence of any element(s) not specifically disclosed herein. Thus, for example, in each instance herein any of the terms “comprising”, “consisting essentially of”, and “consisting of” may be replaced with either of the other two terms.

### PF-07265028 Free Base (Form 2)

In one aspect, the disclosure provides PF-07265028 free base (Form 2). The methods described herein provide PF-07265028 free base (Form 2) which is substantially pure.

As described herein, Form 2 was characterized by PXRD, Raman spectroscopy, and ¹³C solid state NMR spectroscopy. Such crystalline forms may be further characterized by additional techniques, such as Fourier-Transform InfraRed Spectroscopy (FTIR), Differential Scanning Calorimetry (DSC), Thermogravimetric Analysis (TGA) or Differential Thermal Analysis (DTA).

In some embodiments of each of the aspects of the disclosure, PF-07265028 free base (Form 2) is characterized by its powder X-ray diffraction (PXRD) pattern. In other embodiments of each of the aspects of the disclosure, PF-07265028 free base (Form 2) is characterized by its Raman spectrum. In other embodiments of each of the aspects of the disclosure, PF-07265028 free base (Form 2) is characterized by its ¹³C solid state NMR spectrum.

In further embodiments, PF-07265028 free base (Form 2) is characterized by a combination of two or three of these methods. Exemplary combinations including two or more of the following are provided herein: powder X-ray diffraction (PXRD) pattern (2θ); Raman spectrum wavenumber values (cm⁻¹); or ¹³C solid state NMR spectrum (ppm).

It will be understood that any technique described herein and any combination of two or three of these techniques may be used to uniquely characterize PF-07265028 free base (Form 2). In some embodiments PF-07265028 free base (Form 2) is characterized by PXRD and Raman. In some embodiments PF-07265028 free base (Form 2) is characterized by PXRD and ¹³C solid state NMR. In some embodiments PF-07265028 free base (Form 2) is characterized by Raman and ¹³C solid state NMR. In some embodiments PF-07265028 free base (Form 2) is characterized by PXRD, Raman and ¹³C solid state NMR.

In some embodiments, PF-07265028 free base (Form 2) has a PXRD pattern comprising one or more peaks at 2θ values selected from the group consisting of: 9.1, 11.6, 16.7 and 18.5°±0.2°. In some embodiments, PF-07265028 free base (Form 2) has a PXRD pattern comprising two or more peaks at 2θ values selected from the group consisting of: 9.1, 11.6, 16.7 and 18.5°±0.2°. In some embodiments, PF-07265028 free base (Form 2) has a PXRD pattern comprising three or more peaks at 2θ values selected from the group consisting of: 9.1, 11.6, 16.7 and 18.5°±0.2°.

In one embodiment, PF-07265028 free base (Form 2) has a PXRD pattern comprising peaks at 2θ values of: 9.1, 11.6 and 18.5°±0.2°. In some such embodiments, Form 2 has a PXRD pattern further comprising a peak at the 2θ value of: 16.7°±0.2°.

In some embodiments, PF-07265028 free base (Form 2) has a PXRD pattern comprising a peak at a 2θ value of: 9.1°±0.2°. In some embodiments, Form 2 has a PXRD pattern comprising a peak at a 2θ value of: 11.6°±0.2°. In some embodiments, Form 2 has a PXRD pattern comprising a peak at a 2θ value of: 16.7°±0.2°. In some embodiments, Form 2 has a PXRD pattern comprising a peak at a 2θ values of: 18.5°±0.2°. In some such embodiments, the PXRD pattern further comprises one or more additional peaks at 2θ values selected from the group consisting of the peaks in Table 2.

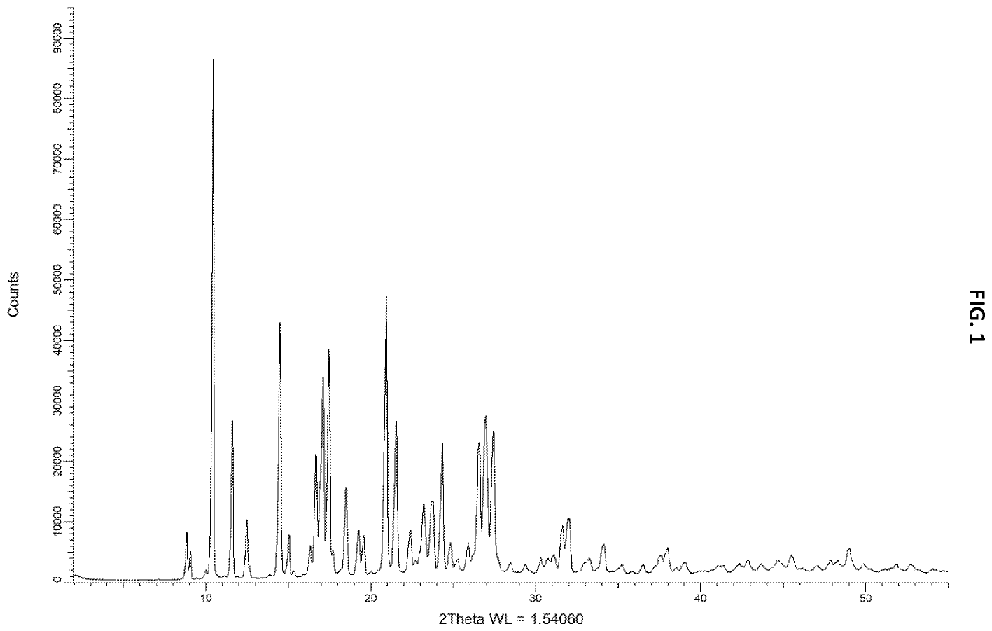

In specific embodiments, PF-07265028 free base (Form 2) has a PXRD pattern comprising: (a) one, two, three, four, five, or more than five peaks selected from the group consisting of the peaks in Table 2 in ° 2θ±0.2° 2θ; or (b) peaks at 2θ values essentially the same as shown in FIG. 1.

In one embodiment, PF-07265028 free base (Form 2) has a Raman spectrum comprising one or more wavenumber (cm⁻¹) values selected from the group consisting of: 1700, 1402, 2827, 1441, and 2996 cm⁻¹±3 cm⁻¹. In another embodiment, PF-07265028 free base (Form 2) has a Raman spectrum comprising two or more wavenumber (cm⁻¹) values selected from the group consisting of: 1700, 1402, 2827, 1441, and 2996 cm⁻¹±3 cm⁻¹. In another embodiment, PF-07265028 free base (Form 2) has a Raman spectrum comprising three or more wavenumber (cm⁻¹) values selected from the group consisting of: 1700, 1402, 2827, 1441, and 2996 cm⁻¹±3 cm⁻¹.

In another embodiment, PF-07265028 free base (Form 2) has a Raman spectrum comprising wavenumber (cm⁻¹) values of: 1700, 1402, and 2827 cm⁻¹±3 cm⁻¹. In some such embodiments, Form 2 has a Raman spectrum further comprising a wavenumber (cm⁻¹) value of: 1441 cm⁻¹±3 cm⁻¹. In some such embodiments, Form 2 has a Raman spectrum further comprising a wavenumber (cm⁻¹) value of: 2996 cm⁻¹±3 cm⁻¹. In another embodiment, PF-07265028 free base (Form 2) has a Raman spectrum comprising wavenumber (cm⁻¹) values of: 1700, 1402, 2827, 1441, and 2996 cm⁻¹±3 cm⁻¹. In another embodiment, PF-07265028 free base (Form 2) has a Raman spectrum comprising a wavenumber (cm⁻¹) value of: 1700 cm⁻¹±3 cm⁻¹. In another embodiment, Form 2 has a Raman spectrum comprising a wavenumber (cm⁻¹) value of: 1402 cm⁻¹±3 cm⁻¹. In another embodiment, Form 2 has a Raman spectrum comprising a wavenumber (cm⁻¹) value of: 2827 cm⁻¹±3 cm⁻¹. In some such embodiments, Form 2 has a Raman spectrum further comprising the wavenumber (cm⁻¹) value of: 1441 cm⁻¹±3 cm⁻¹. In some such embodiments, Form 2 has a Raman spectrum further comprising the wavenumber (cm⁻¹) value of: 2996 cm⁻¹±3 cm⁻¹.

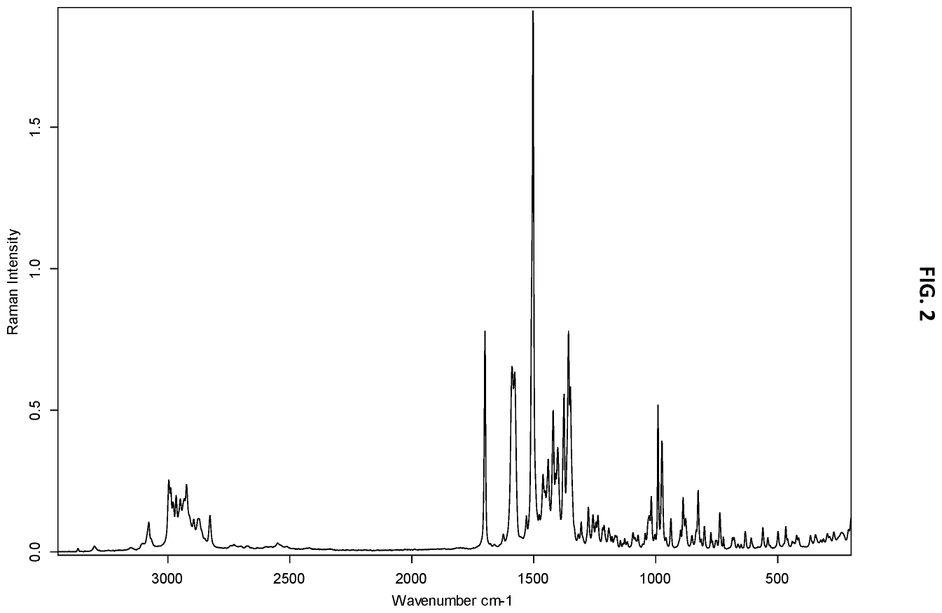

In specific embodiments, PF-07265028 free base (Form 2) has a Raman spectrum comprising: (a) one, two, three, four, five, or more than five wavenumber (cm⁻¹) values selected from the group consisting of the values in Table 3 in cm⁻¹±3 cm⁻¹; or (b) wavenumber (cm⁻¹) values essentially the same as shown in FIG. 2.

In one embodiment, PF-07265028 free base (Form 2) has a ¹³C solid state NMR spectrum comprising one or more resonance (ppm) values selected from the group consisting of: 159.5, 145.8, 152.6, 119.8 and 168.1 ppm±0.2 ppm. In another embodiment, PF-07265028 free base (Form 2) has a ¹³C solid state NMR spectrum comprising two or more resonance (ppm) values selected from the group consisting of: 159.5, 145.8, 152.6, 119.8 and 168.1 ppm±0.2 ppm. In another embodiment, PF-07265028 free base (Form 2) has a ¹³C solid state NMR spectrum comprising three or more resonance (ppm) values selected from the group consisting of: 159.5, 145.8, 152.6, 119.8 and 168.1 ppm±0.2 ppm.

In other embodiments, PF-07265028 free base (Form 2) has a ¹³C solid state NMR spectrum comprising the resonance (ppm) values of: 159.5, 145.8 and 152.6 ppm±0.2 ppm. In some embodiments, PF-07265028 free base (Form 2) has a ¹³C solid state NMR spectrum comprising the resonance (ppm) value of: 159.5 ppm±0.2 ppm. In another embodiment, Form 2 has a ¹³C solid state NMR spectrum comprising the resonance (ppm) value of: 145.8 ppm±0.2 ppm. In another embodiment, Form 2 has a ¹³C solid state NMR spectrum comprising the resonance (ppm) value of: 152.6 ppm±0.2 ppm. In another embodiment, Form 2 has a ¹³C solid state NMR spectrum further comprising the resonance (ppm) value of: 119.8 ppm±0.2 ppm. In another embodiment, Form 2 has a ¹³C solid state NMR spectrum further comprising the resonance (ppm) value of: 168.1 ppm±0.2 ppm.

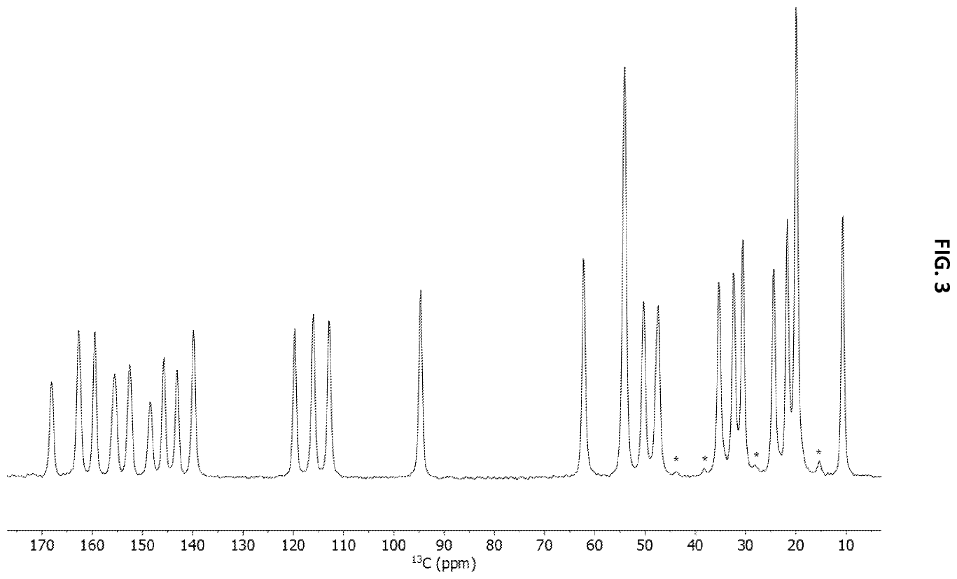

In specific embodiments, PF-07265028 free base (Form 2) has a ¹³C solid state NMR spectrum (ppm) comprising: (a) one, two, three, four, five, or more than five resonance (ppm) values selected from the group consisting of the values in Table 4 in ppm±0.2 ppm; or (b) resonance (ppm) values essentially the same as shown in FIG. 3.

In further embodiments, PF-07265028 free base (Form 2) is characterized by a combination of two, three or four of the embodiments described above that are not inconsistent with each other. Exemplary embodiments that may be used to uniquely characterize Form 2 of PF-07265028 free base are provided below.

In one embodiment, PF-07265028 free base (Form 2) has a powder X-ray diffraction pattern comprising peaks at 2θ values of: 9.1, 11.6, and 18.5°±0.2°.

In another embodiment, PF-07265028 free base (Form 2) has a powder X-ray diffraction pattern comprising peaks at 2θ values of: 9.1, 11.6, 16.7, and 18.5°±0.2°.

In another embodiment, PF-07265028 free base (Form 2) has: (a) a powder X-ray diffraction pattern comprising peaks at 2θ value of: 9.1, 11.6, and 18.5°±0.2°; and (b) a Raman spectrum comprising wavenumber (cm⁻¹) values of: 1700, 1402, and 2827 (±3 cm⁻¹).

In another embodiment, PF-07265028 free base (Form 2) has: (a) a powder X-ray diffraction pattern comprising peaks at 2θ values of: 9.1, 11.6, and 18.5°±0.2°; and (b) a ¹³C solid state NMR spectrum comprising resonance (ppm) values of: 159.5, 145.8 and 152.6 ppm±0.2 ppm.

In another embodiment, PF-07265028 free base (Form 2) has: (a) a Raman spectrum comprising wavenumber (cm⁻¹) values of: 1700, 1402, and 2827 (±3 cm⁻¹); and (b) a ¹³C solid state NMR spectrum comprising resonance (ppm) values of: 159.5, 145.8 and 152.6 ppm±0.2 ppm.

In another embodiment, PF-07265028 free base (Form 2) has: (a) a powder X-ray diffraction pattern comprising peaks at 2θ value of: 9.1, 11.6, and 18.5°±0.2°; (b) a Raman spectrum comprising wavenumber (cm⁻¹) values of: 1700, 1402, and 2827 (±3 cm⁻¹); and (c) a ¹³C solid state NMR spectrum comprising resonance (ppm) values of: 159.5, 145.8 and 152.6 ppm±0.2 ppm.

In another embodiment, PF-07265028 free base (Form 2) has: (a) a powder X-ray diffraction pattern comprising peaks at 2θ value of: 9.1, 11.6, 16.7, and 18.5°±0.2°; and (b) a Raman spectrum comprising wavenumber (cm⁻¹) values of: 1700, 1402, and 2827 cm⁻¹±3 cm⁻¹.

In another embodiment, PF-07265028 free base (Form 2) has: (a) a powder X-ray diffraction pattern comprising peaks at 2θ values of: 9.1, 11.6, 16.7, and 18.5°±0.2°; and (b) a ¹³C solid state NMR spectrum comprising resonance (ppm) values of: 159.5, 145.8 and 152.6 ppm±0.2 ppm.

In another embodiment, PF-07265028 free base (Form 2) has: (a) a powder X-ray diffraction pattern comprising peaks at 2θ value of: 9.1, 11.6, 16.7, and 18.5°±0.2°; (b) a Raman spectrum comprising wavenumber (cm⁻¹) values of: 1700, 1402, and 2827 cm⁻¹±3 cm⁻¹; and (c) a ¹³C solid state NMR spectrum comprising resonance (ppm) values of: 159.5, 145.8 and 152.6 ppm±0.2 ppm.

In another embodiment, PF-07265028 free base (Form 2) has: (a) a Raman spectrum comprising wavenumber (cm⁻¹) values of: 1700, 1402, and 2827 cm⁻¹±3 cm⁻¹; and a ¹³C solid state NMR spectrum comprising resonance (ppm) values of: 159.5, 145.8 and 152.6 ppm±0.2 ppm.

In another embodiment, PF-07265028 free base (Form 2) has: (a) a powder X-ray diffraction pattern comprising peaks at 2θ values of: 9.1, 11.6, 16.7, and 18.5°±0.2°; (b) a Raman spectrum comprising wavenumber (cm⁻¹) values of: 1700, 1402, and 2827 cm⁻¹±3 cm⁻¹; and (c) a ¹³C solid state NMR spectrum comprising resonance (ppm) values of: 159.5, 145.8 and 152.6 ppm±0.2 ppm.

In another embodiment, PF-07265028 free base (Form 2) has a Raman spectrum comprising wavenumber (cm⁻¹) values of: 1700, 1402, 2827 (±3 cm⁻¹), 1441, and 2996 cm⁻¹±3 cm⁻¹.

In another embodiment, PF-07265028 free base (Form 2) has a Raman spectrum comprising wavenumber (cm⁻¹) values of: 1700, 1402, 2827 (±3 cm⁻¹), and 1441 cm⁻¹±3 cm⁻¹.

In another embodiment, PF-07265028 free base (Form 2) has a ¹³C solid state NMR spectrum comprising resonance (ppm) values of: 159.5, 145.8, 152.6 and 119.8 ppm±0.2 ppm.

In another embodiment, PF-07265028 free base (Form 2) has a ¹³C solid state NMR spectrum comprising resonance (ppm) values of: 159.5, 145.8, 152.6, 119.8 and 168.1 ppm±0.2 ppm.

In another aspect, the disclosure provides PF-07265028 free base (Form 2), having: (a) a powder X-ray diffraction (PXRD) pattern comprising peaks at 2θ values of: 9.1, 11.6, and 18.5°±0.2°; (b) a Raman spectrum comprising one or more wavenumber (cm⁻¹) values selected from the group consisting of: 1700, 1402, and 2827 (±3 cm⁻¹); or (c) a ¹³C solid state NMR spectrum comprising one or more resonance (ppm) values selected from the group consisting of: 159.5, 145.8 and 152.6 ppm±0.2 ppm; or a combination of two or more of (a), (b), (c) and (d). Each of the embodiments described herein for individual methods of characterization may be combined with or further limit this aspect, provided the embodiments are not inconsistent with each other.

### Method of Preparing Crystalline Form of PF-07265028 Free Base (Form 2)

In another aspect, the disclosure provides a method of preparing a crystalline Form 2 of PF-07265028 free base, the method comprising the steps of:
providing PF-07265028 free base in a first solvent to form a first solvent solution;
adding the first solvent solution to a second solvent at a temperature from about 73° C. to about 90° C. to form a first-second-solvent solution;
removing proportion of the solvent from the first-second-solvent solution while maintaining a ratio (v/v) of the first solvent to the second solvent in the range from about 20:80 to about 2:98 to obtain a slurry; and
cooling the slurry to obtain the crystalline Form 2 of PF-07265028 free base.

In the above method, PF-07265028 free base is prepared as described herein. In the above method, the addition of the first solvent solution to the second solvent may be performed during a distillation (e.g., azeotropic distillation) at a temperature above the boiling point of the first solvent. The distillation temperature may be set at from about 73° C. to about 90° C., or from about 75° C. to about 85° C., or at about 80° C. In some embodiments, the first solvent solution is added continuously to the second solvent at a steady rate; in some embodiments, the addition is intermittent. The above described adding of the first solvent solution to the second solvent step may be performed simultaneously together with the removing proportion of the solvent from the first-second-solvent solution step. The first solvent may be continuously being removed from the first-second-solvent solution during the distillation. The second solvent may also be removed during the distillation, but to a lesser amount as compared to the amount of the first solvent being removed. The volume ratio of the first solvent to the second solvent in the first-second-solvent solution may varies from time to time but should be kept in the range of from about 20:80 to about 2:98 to obtain the crystalline Form 2 of PF-07265028 free base exclusively. It is critical that the volume ratio of the first solvent to the second solvent is kept in the range of from about 20:80 to about 2:98, in embodiments, from about 15:85 to about 5:95. During the distillation, if the amount in volume of the first solvent is more than 20% based on the total volume of the solvents (i.e., sum of the first solvent volume and the second solvent volume), the process may provide a mixture of different crystalline forms of PF-07265028 free base or the process may provide a different crystalline form other than Form 2 of PF-07265028 free base. For example, the inventors have observed that Form 1 of PF-07265028 free base was formed when the ratio (v:v) of the first solvent to the second solvent is from about 30:70 to about 100:0.

The distillation may be terminated when at least 90% of the first solvent has been removed, and thereby forming a slurry that contains PF-07265028 free base. The slurry may be heated at a temperature of from about 73° C. to about 90° C., or from about 75° C. to about 85° C., or at about 80° C., for a period of time, for example, for 0 to about 2 hours, or for about 15 minutes to about 1 hour. The slurry may then be cooled down to obtain the crystalline form of PF-07265028 free base. Cooling down means bringing the temperature down to a lower temperature than the heating temperature, for example, by 10° C., 20° C., 30° C., 40° C., 50° C., 60° C., 70° C., 80° C., or bringing the temperature down to about 20° C., about 25° C., about 30° C., about 35° C., about 40° C., about 45° C., or about 50° C.

In some embodiments, the first solvent is a solvent having a low boiling point, for example, below 60° C. Examples of the first solvent include dichloromethane, ethyl ether, acetone, and mixtures thereof. In one embodiment, the first solvent is dichloromethane.

In some embodiments, the second solvent is selected from isopropyl acetate, n-propyl acetate, isobutyl acetate, t-butyl acetate, and mixtures thereof. In one embodiment, the second solvent is isopropyl acetate. The inventors have discovered that only a small selection of second solvents are suitable for use in the present method. Not any organic solvent may be able to provide a suitable media to produce the crystalline Form 2 of PF-07265028 free base. For example, the common organic solvent ethyl acetate generates acetylated impurities of the PF-07265028 free base and is not suitable for use as a second solvent in the present method. Without being bound by any theory, it is believed that isopropyl acetate has the capacity to form azeotropes with water, thus providing an anhydrous form of the PF-07265028 free base.

In some embodiments, the method of preparing a crystalline Form 2 of PF-07265028 free base described above comprises:
reacting Compound 8

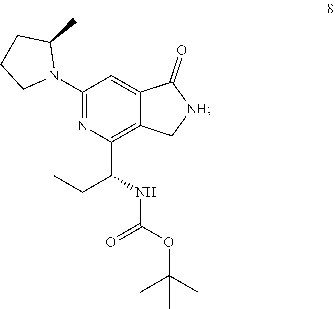 [SMILES: CC[C@@H](NC(=O)OC(C)(C)C)c1nc(N2CCC[C@H]2C)cc2c1CNC2=O]

with Compound 9

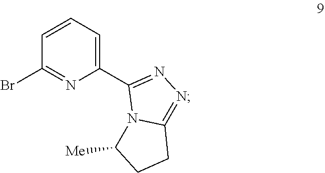 [SMILES: C[C@H]1CCc2nnc(-c3cccc(Br)n3)n21]

in the presence of an organopalladium catalyst to produce Compound 10

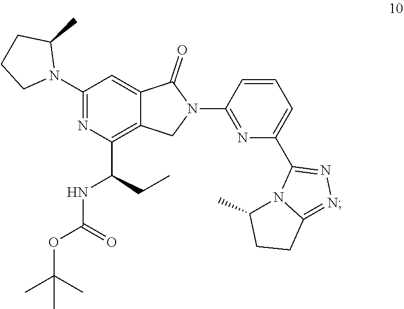 [SMILES: CC[C@@H](NC(=O)OC(C)(C)C)c1nc(N2CCC[C@H]2C)cc2c1CN(c1cccc(-c3nnc4n3[C@@H](C)CC4)n1)C2=O]

deprotecting the amino side chain protecting group in Compound 10 to obtain HCl salt of PF-07265028 of Compound 11

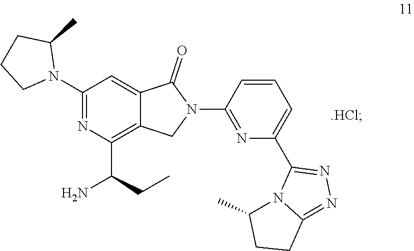

contacting HCl salt of PF-07265028 in a first solvent with a basic solution having a basic pH to obtain a mixture that comprises PF-07265028 free base;
isolating the first solvent that comprises PF-07265028 free base from the mixture;
adding the first solvent that contains PF-07265028 free base to a second solvent at a temperature from about 73° C. to about 90° C. to form a first-second-solvent solution;
removing proportion of the solvent from the first-second-solvent solution while maintaining a ratio (v/v) of the first solvent to the second solvent in the range from about 20:80 to about 2:98 to obtain a slurry; and
cooling the slurry to obtain the crystalline form of PF-07265028 free base.

In some embodiments, the reaction of Compound 8 and Compound 9 may be performed in the presence of an organopalladium catalyst, such as tris(dibenzylideneacetone)dipalladium (0), tetrakis(triphenylphosphine)palladium (0) and/or bis(triphenylphosphine)palladium (II) dichloride. In some embodiments, the organopalladium catalyst is a palladium (0) catalyst. The amount of organopalladium catalyst is from about 0.025 to about 0.2 molar equivalent (equiv) based on the amount of Compound 8 or Compound 9. In some embodiments, the contacting of Compound 8 with Compound 9 is performed at a temperature from about 70° C. to about 100° C.

The deprotecting step to remove the protecting group, e.g., t-butyloxycarbonyl (Boc) in Compound 10 may be performed by contacting Compound 10 with hydrochloric acid. During the process of the deprotecting step, a HCl salt of PF-07265028 of Compound 11 may be produced. In some embodiments, the amount of hydrochloric acid is from about 10 to about 20 molar equivalents based on the amount of Compound 10. In some embodiments, the concentration of hydrochloric acid is from about 1 N to about 6 N, or about 6 N. In some embodiments, the contacting of Compound 10 with hydrochloric acid is carried out for at least 10 hours, for example, from about 12 to about 25 hours, or from about 15 to about 20 hours. Upon the completion of the deprotecting step, the HCl salt of PF-07265028 of Compound 11 produced may be suspended in an organic solvent, such as acetone, tetrahydrofuran or 2-methyl tetrahydrofuran, and may be filtered through to remove any dissolved impurities. By removal of said organic solvent by filtration, the HCl salt of PF-07265028 of Compound 11 may be obtained in solid form.

The HCl salt of PF-07265028 of Compound 11 may be dissolved in the first solvent. Contacting the HCl salt of PF-07265028 of Compound 11 in the first solvent with a basic solution having a basic pH may produce a mixture that comprises PF-07265028 free base. In some embodiments, the basic solution having a basic pH value of from about 8 to about 14, or from about 9 to about 11. In some embodiments, the basic solution comprises a strong base such as sodium hydroxide, potassium hydroxide, sodium carbonate, or mixtures thereof. In some embodiments, the method comprises isolating the first solvent that comprises PF-07265028 free base from the mixture. The isolating may simply be performed by separating the organic layer and the aqueous layer of the mixture. The organic layer from the previous step contains the first solvent that comprises PF-07265028 free base may be combined with a second solvent. The combined first-second-organic-solvent solution may be contacted with a thiol functionalized silica gel to remove any excess metal. In some embodiments, the contacting with the thiol functionalized silica gel is performed at a temperature of from about 35° C. to about 48° C. for about 12-18 hours.

In another aspect, the disclosure provides a pharmaceutical composition comprising the crystalline form of PF-07265028 free base (Form 2) according to any of the embodiments described herein, and a pharmaceutically acceptable carrier or excipient.

In another aspect, the disclosure provides method of treating abnormal cell growth in a mammal, preferably a human, comprising administering to the mammal a therapeutically effective amount of the crystalline form of PF-07265028 free base (Form 2) according to any of the embodiments described herein.

In another aspect, the disclosure provides method of treating abnormal cell growth in a mammal, preferably a human, comprising administering to the mammal a therapeutically effective amount of a pharmaceutical composition comprising the crystalline form of PF-07265028 free base (Form 2) according to any of the embodiments described herein.

In another aspect, the disclosure provides the crystalline form of PF-07265028 free base (Form 2) according to any of the embodiments described herein for use in treating abnormal cell growth in a mammal, such as, a human.

In another aspect, the disclosure provides the use of the crystalline form of PF-07265028 free base (Form 2) according to any of the embodiments described herein in treating abnormal cell growth in a mammal, such as, a human.

In another aspect, the disclosure provides use of the crystalline form of PF-07265028 free base (Form 2) according to any of the embodiments described herein in the manufacture of a medicament for use in a treating abnormal cell growth in a mammal, such as, a human.

In frequent embodiments of the methods, compositions and uses described herein, the abnormal cell growth is cancer.

Pharmaceutical compositions of the present disclosure may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the disclosure as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

### EXAMPLES

The examples and preparations provided below further illustrate and exemplify particular aspects and embodiments of the disclosure. It is to be understood that the scope of the present disclosure is not limited by the scope of the following examples.

### General Method 1. Powder X-Ray Diffraction (PXRD)

### Instrument Method:

Powder X-ray diffraction analysis was conducted using a Bruker AXS D8 Endeavor diffractometer equipped with a copper (Cu) radiation source, nickel filter, and theta-theta goniometer. The tube voltage and amperage were set to 40 kV and 40 mA, respectively, and the motorized divergence slits were set at constant illumination of 11 mm. Diffracted x-ray radiation was detected using a LYNXEYE XE-T energy dispersive x-ray detector, with the position sensitive detector (PSD) opening set at 4.00°. A sample was placed in a silicon low background small divot holder and smoothed with a glass slide. The powder pattern was collected at room temperature using Cu Kα1 (λ=1.5406 Å) radiation from 2.0° to 55.0° (2θ) with a step size of 0.019°, time per step of 0.2 s, and 15 rpm.

### Peak Picking Method:

Data was imported and analyzed in DIFFRAC.EVA V5.0 software. The collected powder pattern of PF-07265028 Form 2 was aligned to the simulated powder pattern from the crystal structure obtained at room temperature. An initial list of peaks was generated using the Peak Search function in EVA with a width of 0.200 and threshold of 1.0. From this a shortened list was prepared using those peaks with a relative intensity ≥5% of the most intense peak. A typical variability of ±0.2° (2θ) in peak positions (USP-941) applies to this data.

### General Method 2. Raman Spectroscopy

### Instrument Method:

Raman spectra were collected using a Bruker RAM II FT-Raman module attached to a Vertex 70 spectrometer. The instrument is equipped with a 1064 nm solid-state (Nd:YAG) laser and a liquid nitrogen cooled germanium detector. Prior to data acquisition, instrument performance and calibration verifications were conducted using a white light source, and polystyrene and naphthalene references.

Samples were prepared and analyzed in truncated NMR tubes. A sample rotator (Ventacon, UK) was used during measurement to maximize the volume of material analyzed during data collection, to minimize relative intensity variations caused by preferred orientation. The backscattered Raman signal from the sample collected at a spectral resolution of 3 cm⁻¹ using a laser power of 500 mW. A Blackmann-Harris 4-term apodization function was applied to minimize spectral aberrations. Spectra were generated between 3500 and 50 cm⁻¹ with the number of scans adjusted accordingly to ensure adequate signal to noise.

### Peak Picking Method:

Spectra were normalized to the intensity of the most intense peak. Peak positions and relative intensities were obtained using the automatic peak picking function in the OPUS v8.2 software with the sensitivity set to 2.0%. The variability in the peak positions with this experimental configuration is within ±3 cm⁻¹, unless otherwise stated.

It is expected that, since FT-Raman and dispersive Raman are similar techniques, peak positions reported in this document for FT-Raman spectra would be consistent with those which would be observed using a dispersive Raman measurement, assuming appropriate instrument calibration.

The characteristic peaks for these forms were chosen based on their intensity, as well as peak position.

### General Method 3. Solid State NMR (ssNMR) Spectroscopy

### Instrument Method:

Solid state NMR analysis was conducted on a Bruker AVANCE NEO 400 MHz (¹H frequency) NMR spectrometer using a 4 mm MAS probe at a magic angle spinning (MAS) rate of 12.5 kHz. The temperature was 20° C. in each case and a phase modulated proton decoupling field of 80-100 kHz was applied during spectral acquisition.

¹³C cross-polarization (CP) spectra were recorded with a 1.75 s CP contact time and recycle delay of 3 s. Carbon spectral referencing is relative to neat tetramethylsilane, carried out by setting the high-frequency signal from an external sample of L-alanine to 177.8 ppm.

### Peak Picking Method:

Peak positions and relative intensities were obtained using ACD Labs 2020 Spectrus Processor software with the threshold for peak selection set to 3% relative intensity. The output of the automated peak picking was visually checked to ensure validity and adjustments were made if necessary. Generally, a variability of ±0.2 ppm applies to the reported ¹³C chemical shifts.

Although specific solid-state NMR peak values are reported herein there does exist a range for these peak values due to differences in instruments, samples, and sample preparation. This is common practice in the art of solid-state NMR because of the variation inherent in peak positions. A typical variability for a ¹³C chemical shift x-axis value is on the order of plus or minus 0.2 ppm for a crystalline solid. The solid-state NMR peak heights reported herein are relative intensities. Solid state NMR intensities can vary depending on the actual setup and the thermal history of the sample.

### Example 1

Synthesis of Compound 8: tert-butyl ((R)-1-(6-((R)-2-methylpyrrolidin-1-yl)-1-oxo-2,3-dihydro-1H-pyrrolo[3,4-c]pyridin-4-yl)propyl)carbamate

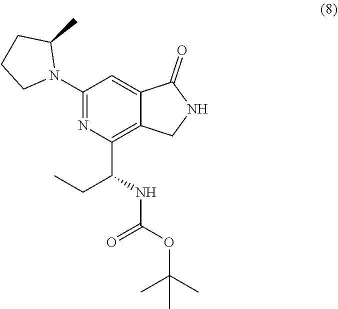 [SMILES: CC[C@@H](NC(=O)OC(C)(C)C)c1nc(N2CCC[C@H]2C)cc2c1CNC2=O]

Compound 8 was synthesized according to the synthetic scheme shown below:

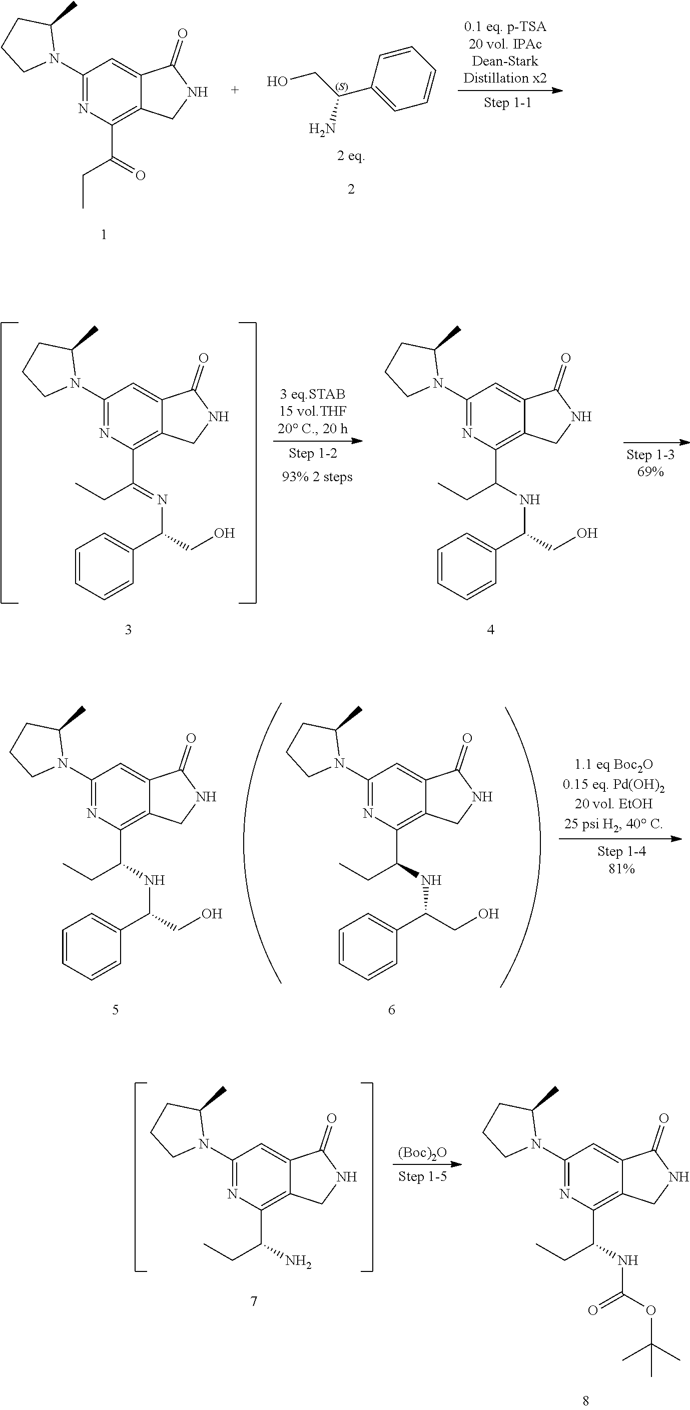

STEP 1-1: 4-((E)-1-(((S)-2-hydroxy-1-phenylethyl)imino)propyl)-6-((R)-2-methylpyrrolidin-1-yl)-2,3-dihydro-1H-pyrrolo[3,4-c]pyridin-1-one (Compound 3)

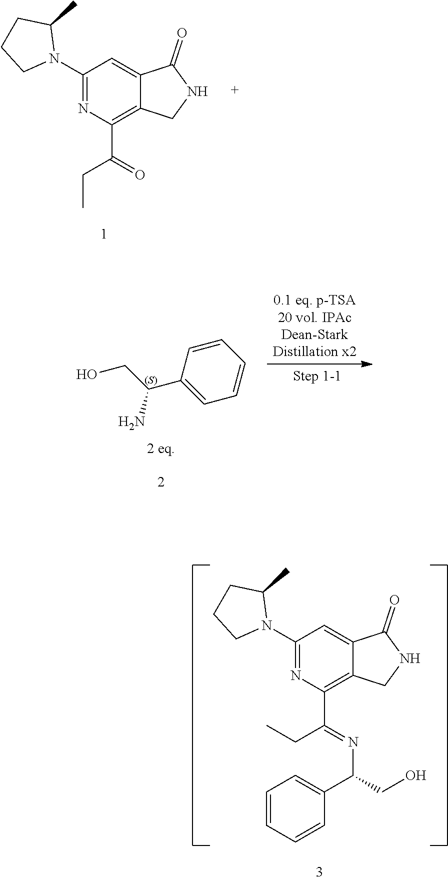

In a reactor provided with a stir bar and a Dean-Stark trap, to a solution of Compound 1 (5 g, 18.29 mmol) (obtained from WuXi STA Co.,Ltd. Shanghai, China) and Compound 2 (5.019 g, 36.58 mmol) in isopropyl acetate (20 mL/g), p-toluene sulfonic acid monohydrate (0.3511 g) was charged. The reaction was heated to 100° C. for 20 h ensuring a controlled Dean-Stark distillation. The temperature was increased to 110° C. and the solvent from the trap removed until 5 volumes of solvent remained. A second addition of isopropyl acetate (10 mL/g) was charged to drive the reaction to completion and the distillation was continued until about 3 volumes of solvent remained. A sample of the residual thick solution was analyzed by ¹H-NMR in CDCl₃ (with TMS). The aromatic pyridine nitrogen was used as reference for product formation (Compound 3) vs unreacted ketone (Compound 1). The ¹H NMR chemical shift of Ha and Hb are shown below:

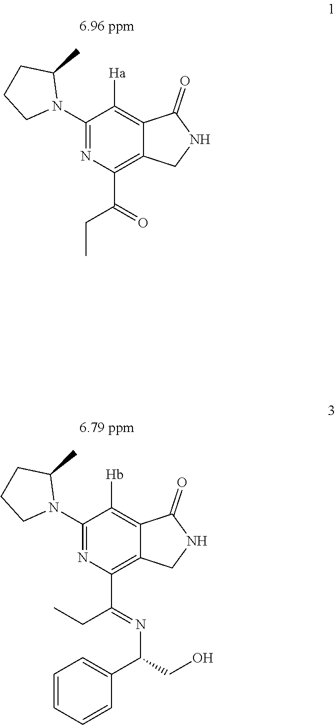

The reactor was cooled to 80° C. and tetrahydrofuran (10 mL/g) was charged. The distillation was resumed until about 5 volumes of solvent were distilled and the solution was telescoped to the next step.

### STEP 1-2: Preparation of Compound 4.

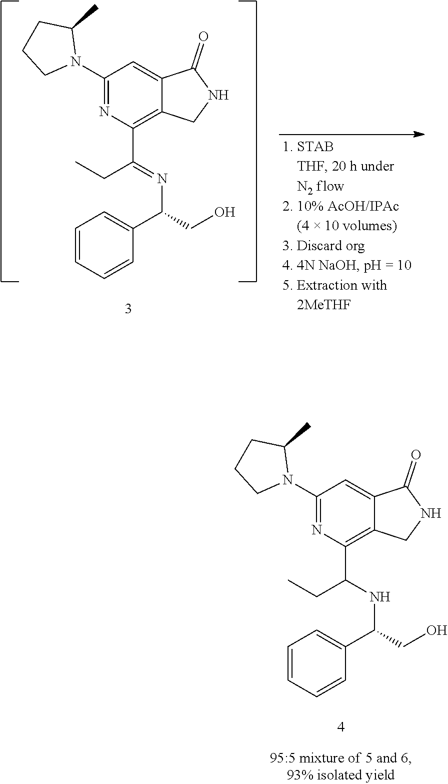

In a 100 mL reactor vessel under a positive flow of nitrogen, sodium triacetoxy borohydride (11.63 g, 3equiv, 54.88 mmol) and tetrahydrofuran (5 mL/g) were charged. The solution from the previous step (Compound 3 in tetrahydrofuran) was charged at 20° C. The suspension was stirred at 20° C. for about 20 h or until the IPC shows complete conversion. Typically, about 5% of unreacted ketone is observed.

Methanol (10.00 equiv, 182.9 mmol, 5.862 g, 7.402 mL) was charged and the reaction was stirred until all the solids dissolved. Acetic acid in water (50 mL, 4.770 equiv, 87.26 mmol) was charged (A slight exotherm and some off-gas was observed) and the reaction stirred for 1 h.

The solvent was reduced at 40° C. to 10 volumes and isopropyl acetate (10 mL/g) was charged and the reaction stirred for 20 min.

The solution was transferred to a separatory funnel and the layers were allowed to separate. The aqueous layer was transferred to the reactor and the organic layer was extracted with 3×10 vol of 10% acetic acid in water to ensure all the product was extracted from the organic layer as judged by LCMS. The organic layer was discarded, and the aqueous extracts were combined in a flask provided with magnetic stirring. Sodium hydroxide (4 mol/L) in water (15.00 equiv, 274.4 mmol, 68.61 mL) was charged portion wise using an addition funnel to take the solution to pH=10. The solution turned into a biphasic mixture that precipitated after a few minutes of stirring. The reaction was cooled in an ice bath and stirred for 2 h and the solids obtained were filtered using water (75 mL). The crude product was dried in a vacuum oven at 50° C. for 20 h to give a yellow solid.

Alternatively, the product can be extracted with 3×10 volumes of 2-methyl tetrahydrofuran after step 28 and use this solution to crystallize the product in acetone/methyl tert-butyl ether.

### STEP 1-3: Crystallization of Compound 5

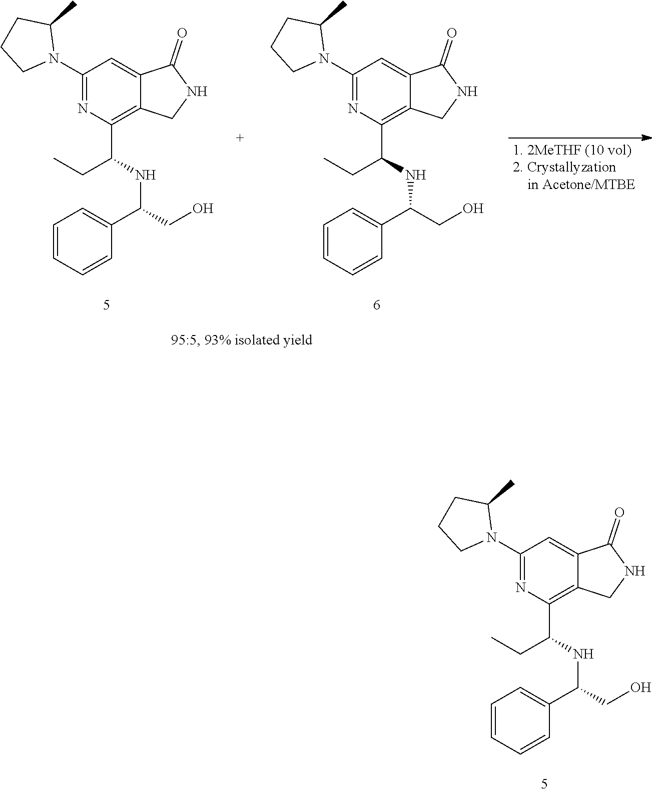 [SMILES: CC[C@@H](N[C@H](CO)c1ccccc1)c1nc(N2CCC[C@H]2C)cc2c1CNC2=O ; CC[C@H](N[C@H](CO)c1ccccc1)c1nc(N2CCC[C@H]2C)cc2c1CNC2=O ; CC[C@@H](N[C@H](CO)c1ccccc1)c1nc(N2CCC[C@H]2C)cc2c1CNC2=O]

A solution of the crude 95:5 mixture of isomers in 2-methyltetrahydrofuran (10 volumes) was charged into a 100 mL reactor provided with overhead stirring. The solution was solvent-exchanged with acetone (10 volumes) and the solvent was reduced until about 5 volumes of acetone remained. The resulting slurry was stirred at 25° for 24 h and methyl tert-butyl ether (10 volumes) was charged. The solvent was reduced under mild vacuum to 5 volumes, the solids were granulated for 2 h and filtered using methyl tert-butyl ether to give Compound 5 as single isomer in >98% purity, 99.26% UPLC purity. M+1=395.24 AMU.

¹H NMR (400 MHz, Chloroform-d) δ 0.82 (t, J=7.46 Hz, 3H), 1.29 (d, J=6.36 Hz, 3H), 1.68-1.86 (m, 3H), 1.97-2.20 (m, 3H), 3.32 (t, J=7.03 Hz, 1H), 3.35-3.43 (m, 1H), 3.46-3.51 (m, 1H), 3.52-3.72 (m, 4H), 4.04 (d, J=15.77 Hz, 1H), 4.25 (br t, J=5.99 Hz, 1H), 6.49 (s, 1H), 6.66 (s, 1H), 7.23-7.27 (m, 2H), 7.27 (s, 2H), 7.28-7.36 (m, 3H)

### STEPS 1-4 & 1-5: Protecting Group Exchange to Compound 8

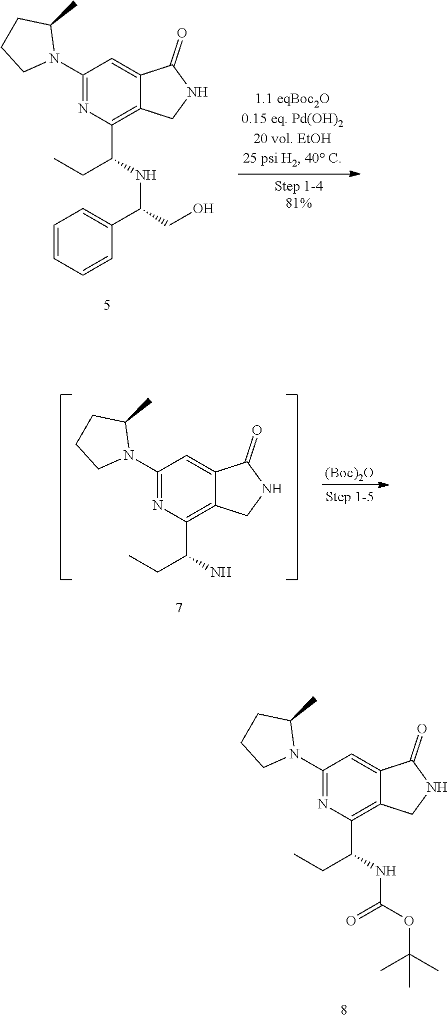

In a hydrogenation reactor, palladium hydroxide (II) (335 mg, 15% mol), Compound 5 (10 g, 25.34 mmol) and (Boc)₂O (6.079 g, 27.87 mmol) in absolute ethanol (200 mL) was charged. The hydrogenation reactor was sealed, pressurized to 25 psi and heated to 40° C., until reaction completion as judged by UPLC. The reaction was filtered into a reactor through filter paper and the solution was concentrated at 65° C. under mild vacuum to about 2 volumes.

The solution was cooled to 45° C. and methyl tert-butyl ether (10 volumes) was charged. The solution was cooled gradually to 20° C. to give a mobile slurry. A second heat/cool cycle to 55° C. for 1 h, followed by gradual cooling to 20° C. provided the product as a crystalline solid. The product was pulled dry under vacuum and dried further in a vacuum oven at 45° C. for 8 h to give the product Compound 8 in >98% purity and yield 75% yield. M+1=375.1 AMU, ¹H NMR (400 MHz, DMSO-d6), 80° C. δ 0.89 (t, J=7.40 Hz, 3H), 1.22 (d, J=6.11 Hz, 3H), 1.37 (s, 9H), 1.66-1.80 (m, 2H), 1.83-2.01 (m, 2H), 2.01-2.18 (m, 2H), 3.32-3.41 (m, 1H), 3.52 (ddd, J=10.21, 7.21, 3.48 Hz, 1H), 4.15-4.24 (m, 1H), 4.24-4.43 (m, 1H), 4.44-4.56 (m, 1H), 6.48 (s, 1H), 6.53-6.66 (m, 1H), 8.34 (br s, 1H).

### Example 2

Synthesis of Compound 9: (5S)-3-(6-bromopyridin-2-yl)-5-methyl-6,7-dihydro-5H-pyrrolo[2,1-c][1,2,4]triazole

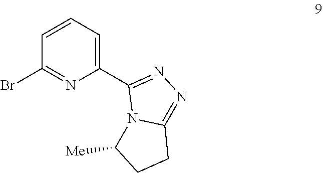 [SMILES: C[C@H]1CCc2nnc(-c3cccc(Br)n3)n21]

Step 2-1: Tert-butyl{(2S)-5-[2-(6-bromopyridine-2-carbonyl)hydrazinyl]-5-oxopentan-2-yl}carbamate 9a

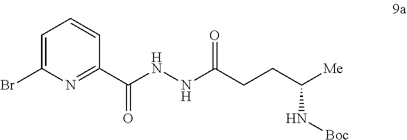 [SMILES: C[C@@H](CCC(=O)NNC(=O)c1cccc(Br)n1)NC(=O)OC(C)(C)C]

A solution of (4S)-4-[(tert-butoxycarbonyl)amino]pentanoic acid (CAS: 207924-92-3) (600 mg, 2.76 mmol) in tetrahydrofuran (13.8 mL, 0.2 M) was cooled to 0° C. Propylphosphonic anhydride solution (50% solution in ethyl acetate, 3.62 mL, 6.08 mmol) was added to the solution at 0° C. before the bath was removed and the reaction mixture was stirred for 30 min at ambient temperature. Then, N,N-diisopropylethylamine (2.89 mL, 16.6 mmol) and 6-bromopicolinohydrazide (656 mg, 3.04 mmol) were added and the reaction mixture was stirred at ambient temperature for 22 h. LCMS analysis showed consumption of the starting material. The reaction was quenched with water (15 mL) and transferred to a separatory funnel with ethyl acetate (20 mL). The layers were separated, and the organic phase was washed sequentially with 20% citric acid (20 mL), a saturated solution of sodium bicarbonate (20 mL), and brine (20 mL). The organic extract was then dried over magnesium sulfate, filtered, and concentrated to dryness to provide the title compound (9a) (1.07 g, 93% yield) as an off-white solid, which was taken on without further purification. ¹H NMR (400 MHz, CDCl₃) δ 9.76 (br. s, 1H), 9.36 (br. s, 1H), 8.15 (dd, J=0.9, 7.5 Hz, 1H), 7.77-7.71 (m, 1H), 7.69-7.64 (m, 1H), 4.45 (br. d, J=1.0 Hz, 1H), 3.91 (br. s, 1H), 2.47-2.35 (m, 2H), 2.00-1.89 (m, 1H), 1.75-1.66 (m, 1H), 1.48 (s, 9H), 1.22 (d, J=6.6 Hz, 3H). LCMS m/z (APCI) for (C₁₁H₁₅BrN₄O₂), 315.0 (M+H-Boc)⁺.

Step 2-2: Tert-butyl{(2S)-4-[5-(6-bromopyridin-2-yl)-1,3,4-oxadiazol-2-yl]butan-2-yl}carbamate 9b

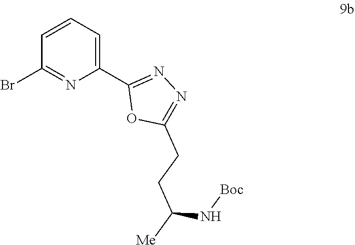 [SMILES: C[C@@H](CCc1nnc(-c2cccc(Br)n2)o1)NC(=O)OC(C)(C)C]

To a solution of tert-butyl {(2S)-5-[2-(6-bromopyridine-2-carbonyl)hydrazinyl]-5-oxopentan-2-yl}carbamate 9a (290 mg, 0.698 mmol) in dichloromethane (2.8 mL, 0.25 M) was added triethylamine (0.292 mL, 2.09 mmol) and p-toluenesulfonyl chloride (160 mg, 0.838 mmol). The reaction was stirred at ambient temperature for 16 h. LCMS analysis showed consumption of the starting material. Ethylenediamine (0.047 mL, 0.698 mmol) was added to scavenge excess p-toluenesulfonyl chloride; during the addition, a precipitate formed immediately. After stirring at RT for 30 min, the reaction was washed with 20% citric acid (5 mL) and the layers were separated. The aqueous layer was extracted with dichloromethane (5 mL), then the combined organic layers were washed with brine (10 mL), dried over magnesium sulfate, filtered and concentrated to dryness to provide the title compound 9b (274 mg, 98% yield) as a light yellow solid, which was taken on without further purification. ¹H NMR (400 MHz, CDCl₃) δ 8.21 (dd, J=0.9, 7.6 Hz, 1H), 7.76-7.69 (m, 1H), 7.68-7.65 (m, 1H), 4.39 (br. s, 1H), 3.85 (br. s, 1H), 3.08-3.01 (m, 2H), 2.17-2.03 (m, 1H), 2.03-1.90 (m, 1H), 1.44 (s, 9H), 1.22 (d, J=6.6 Hz, 3H). LCMS m/z (APCI) for (C₁₁H₁₃BrN₄O), 297.0 (M+H-Boc)⁺.

### Step 2-3: Compound 9

A microwave vial was charged with tert-butyl {(2S)-4-[5-(6-bromopyridin-2-yl)-1,3,4-oxadiazol-2-yl]butan-2-yl}carbamate 9b (150 mg, 0.378 mmol) and trifluoroethanol (1.89 mL, 0.2 M) and was sealed before heating in the microwave to 180° C. for 30 min. LCMS analysis showed consumption of the starting material. The reaction mixture was concentrated, and the residue was purified by flash chromatography (silica gel, 100% heptane to 1:10 methanol/ethyl acetate) to provide Compound 9 (74.3 mg, 71% yield) as a tan, gummy solid. ¹H NMR (400 MHz, CDCl₃) δ 8.38 (d, J=7.7 Hz, 1H), 7.74 (t, J=7.8 Hz, 1H), 7.58 (d, J=7.9 Hz, 1H), 5.22-5.08 (m, 1H), 3.21-3.16 (m, 1H), 3.16-3.02 (m, 2H), 2.55-2.44 (m, 1H), 1.60 (d, J=6.6 Hz, 3H). LCMS m/z (APCI) for (C₁₁H₁₁BrN₄), 279.1 (M+H)⁺. Determined to be a single enantiomer by SFC (10-60% methanol (0.5% NH₃) in carbon dioxide at 400-450 bar, gradient time=2 min, flow rate=4 mL/min, Chiralpack IC-U 50 mm*3 mm*1.6 μm column). Stereochemistry of Compound 9 was assigned based on use of (4S)-4-[(tert-butoxycarbonyl)amino]pentanoic acid in step 1.

Absolute configuration of Compound 9 was unambiguously established by small molecule X-ray crystallography. The single crystal X-ray diffraction studies were carried out on a Bruker APEX II Ultra CCD diffractometer equipped with Mo K_{α} radiation (λ=0.71073). Crystal were used as received (grown by vapor diffusion from dichloromethane) A 0.200×0.075×0.060 mm colorless crystal was mounted on a Cryoloop with Paratone oil. Data were collected in a nitrogen gas stream at 100(2) K) using Φ and ω scans. Crystal-to-detector distance was 45 mm using exposure time 1 s (depending on the 2 θ range) with a scan width of 0.80°. Data collection was 100.0% complete to 25.242° in θ. The data were integrated using the Bruker SAINT software program and scaled using the SADABS software program. Solution by direct methods (SHELXT) produced a complete phasing model consistent with the proposed structure. All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. Positions of the C—H, N—H and O—H hydrogen atoms have been refined using appropriate HFIX commands. Crystallographic data are summarized in Table A.

TABLE A

| Molecular formula | C₁₁H₁₁BrN₄ | |
|---|---|---|
| Formula weight | 279.15 | |
| Temperature | 100.15 K | |
| Wavelength | 0.71073 Å | |
| Crystal system | Monoclinic | |
| Space group | P2₁ | |
| Unit cell dimensions | a = 7.772(2) Å | α = 90°. |
| | b = 13.571(4) Å | β = 96.191(6)°. |
| | c = 10.676(3) Å | γ = 90°. |

| | |
|---|---|
| Volume | 1119.5(5) Å³ |
| Z | 4 |
| Density (calculated) | 1.656 Mg/m³ |
| Absorption coefficient | 3.649 mm⁻¹ |
| F(000) | 560 |
| Crystal size | 0.2 × 0.075 × 0.06 mm³ |
| Crystal color, habit | colorless block |
| Theta range for data | 1.919 to 26.755°. |
| collection | |
| Index ranges | −9 <= h <= 9, −17 <= k <= 17, |
| | −13 <= l <= 13 |
| Reflections collected | 19401 |
| Independent reflections | 4760 [Rᵢₙₜ = 0.0524] |
| Completeness to theta = | 100.0% |
| 25.242° | |
| Absorption correction | Semi-empirical from equivalents |
| Max. and min. transmission | 0.4910 and 0.3848 |
| Refinement method | Full-matrix least-squares on F² |
| Data/restraints/parameters | 4760/1/291 |
| Goodness-of-fit on F² | 1.013 |
| Final R indices [I > 2sigma(I)] | R1 = 0.0243, wR2 = 0.0582 |
| R indices (all data) | R1 = 0.0258, wR2 = 0.0589 |
| Absolute structure parameter | −0.012(6) |
| Largest diff. peak and hole | 0.300 and −0.219 e.Å⁻³ |

### Example 3

Synthesis of PF-07265028: 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one

### Steps 3-1 and 3-2

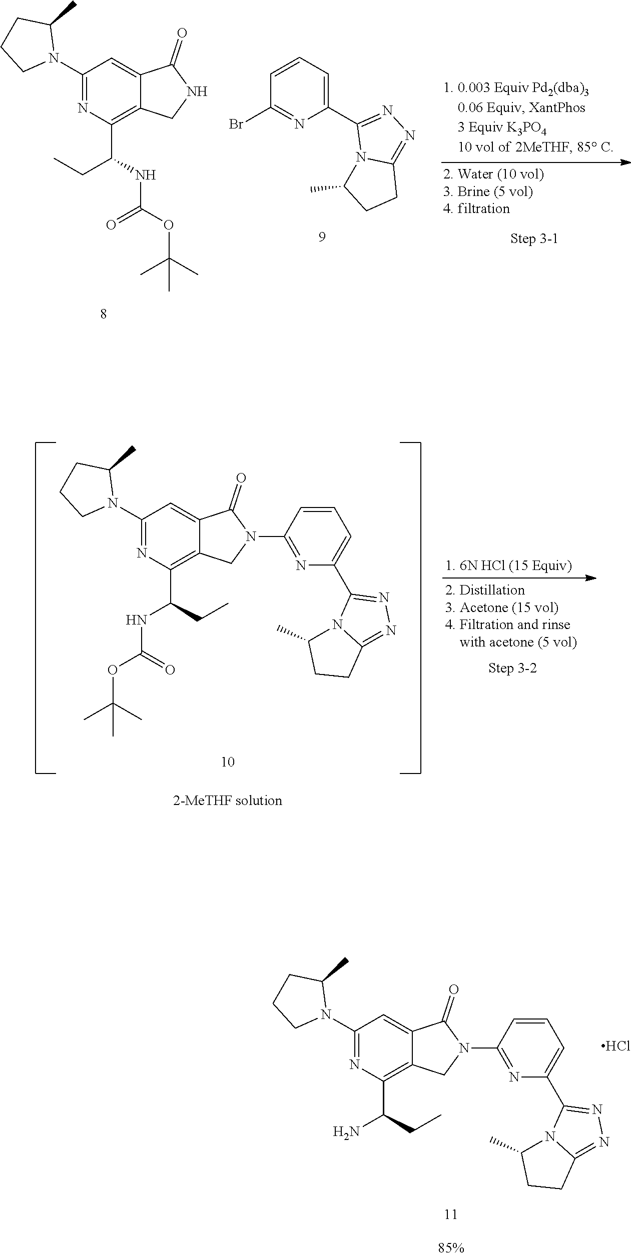

In a reactor equipped with an overhead agitation a nitrogen inlet and a reflux condenser, a solution of Compound 8 (10 g, 26.70 mmol) and Compound 9 (7.8 g, 28.04 mmol) in 2-methyltetrahydrofuran (100 mL) was charged. Potassium phosphate tribasic (17.35 g, 80.11 mmol) and 4,5-bis(diphenylphosphino)-9,9-dimethylxanthene (0.945 g, 1.60 mmol) were charged. The mixture was purged under vacuum and filled with nitrogen (3×5 min purge) before adding tris(dibenzylideneacetone)dipalladium (0) (0.756 g, 0.801 mmol).

The reaction was heated to 85° C. for 18-20 h. Upon reaction completion, water (50 mL) was charged. The reaction mixture was stirred at room temp for 10 min and transferred to a separatory funnel. The aqueous layer was drained, and the organic layer was washed with brine (100 mL), filtered through filter paper and transferred to a reactor. The resulting filtrate was reduced to 5 volumes at 65° C. under reduced pressure and taken to the next step.

At 25° C., hydrochloric acid (6 mol/L) in water (67 mL, 15.00 equiv, 400.5 mmol) was charged and the reaction was stirred at 25° C. for 18-20 h. Upon reaction completion, the resulting slurry was diluted with acetone (150 mL)) and stirred for 2 h. The solids obtained were filtered using acetone (50 mL) and the cake was dried for 20 h in a vacuum oven at 45° C. to give the product 11 as a bright yellow solid (12.3 g, 90%). LCMS m/z for C₂₆H₃₂N₈O M+1=473.1 AMU, ¹H NMR (400 MHz, DMSO-d6) δ 0.96 (t, J=7.46 Hz, 3H), 1.26 (d, J=6.24 Hz, 3H), 1.55 (d, J=6.48 Hz, 3H), 1.76 (dt, J=4.83, 2.60 Hz, 1H), 1.90-2.21 (m, 5H), 2.41-2.48 (m, 1H), 2.92-3.25 (m, 3H), 3.48-3.56 (m, 1H), 3.58-3.69 (m, 1H), 4.22-4.45 (m, 2H), 4.96-5.40 (m, 8H), 6.77 (s, 1H), 8.01 (dd, J=7.58, 0.86 Hz, 1H), 8.10 (t, J=7.95 Hz, 1H), 8.43 (br s, 3H), 8.56 (dd, J=8.31, 0.73 Hz, 1H).

### Steps 3-3 and 3-4

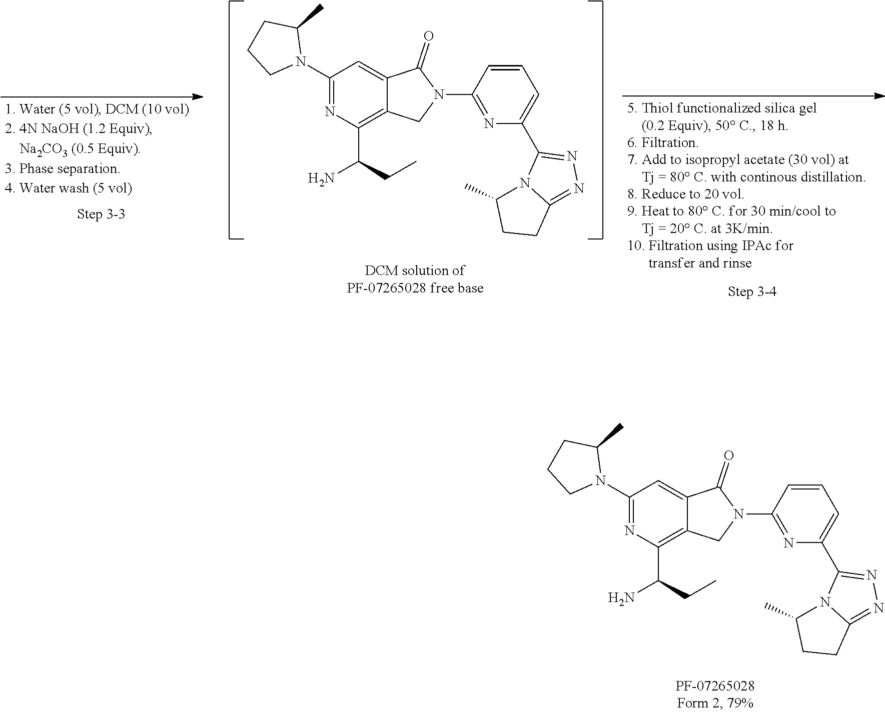

To a 100 mL reactor, compound 11 (5 g, 9.823 mmol), water (34 mL) and dichloromethane (68 mL) were charged, and the mixture was stirred at room temperature until all the materials dissolved. Sodium hydroxide (4 mol/L) in water (12 mL, 48 mmol) was charged to take the pH to 10. The mixture was transferred to a separatory funnel and the layers were allowed to split. The organic layer was drained, and the aqueous layer was extracted with dichloromethane (34 mL). The organics layers were combined, washed with water (5 mL/g, 34 mL×2) and dried over sodium sulfate. The solids were filtered off and the filtrate was concentrated to 10 volumes.

Thiol functionalized silica gel was charged (2.1 g, 0.2 equiv) and the mixture was heated to 45° C. for 18 h. The mixture was filtered through filter paper and transferred to an addition funnel.

Isopropyl acetate (30 mL/g, 150 mL) was charged to a clean reactor provided with a distillation head and a gentle nitrogen sweep. At 80° C., the dichloromethane solution from the previous step was added to the isopropyl acetate while maintaining a constant distillation rate at atmospheric pressure (maintaining constant volume). At the end of the addition, the solvent was reduced at atmospheric pressure to 20 volumes and the resulting slurry was heated to 80° C. for 30 min then cooled gradually to 20° C. The product was granulated for 1 h and filtered using isopropyl acetate to transfer and rinse the cake.

The solids were dried in a vacuum oven at 80° C. for 20 h to give the product as a light-yellow solid, PF-07265028 Form 2 (5 g, 79% Yield). LCMS m/z for C₂₆H₃₂N₈O M+1=473.4 AMU. ¹H NMR (400 MHz, CHLOROFORM-d) ppm=8.60 (d, J=8.4 Hz, 1H), 8.04 (d, J=7.5 Hz, 1H), 7.83 (t, J=8.0 Hz, 1H), 6.63 (s, 1H), 5.09-4.93 (m, 3H), 4.19 (br t, J=5.9 Hz, 1H), 3.80 (t, J=6.7 Hz, 1H), 3.58-3.49 (m, 1H), 3.40-3.26 (m, 1H), 3.12-2.91 (m, 3H), 2.47-2.33 (m, 1H), 2.14-1.92 (m, 3H), 1.85-1.75 (m, 3H), 1.49 (d, J=6.5 Hz, 3H), 1.20 (d, J=6.3 Hz, 3H), 0.88 (t, J=7.4 Hz, 3H).

### Example 4

### Characterization of PF-07265028 Anhydrous Free Base (Form 2)

Form 2 was characterized as a highly crystalline non-hygroscopic anhydrous solid form with a melting point onset of 204° C. Due to its properties, Form 2 appears to be a beneficial form of PF-07265028 from a crystallization, formulation, and manufacturing perspective. Properties of PF-07265028 Form 2 are given in Table 1.

### Table 1. Properties of PF-07265028 Form 2

| Analysis | Form 2 |
|---|---|
| Thermal Analysis | |
| Melting Point (DSC)-Anhydrous | Tₒₙₛₑₜ = ~204° C. |
| Hygroscopicity | |
| Moisture Sorption (DVS)- | ~0.2% at 60% RH |
| Anhydrous | ~0.2% at 75% RH |
| | ~0.2% at 90% RH |
| | Post-DVS PXRD is consistent with in- |
| | going material |

### PXRD Data

FIG. 1 shows PXRD data for PF-07265028 free base (Form 2), collected according to General Method 1. A list of PXRD peaks at diffraction angles ±0.2° (2θ) and their relative intensities is provided in Table 2.

TABLE 2
PXRD Peak List for PF-07265028 free base Form 2

| Angle ± 0.2° (2θ) | Relative Intensity |
|---|---|
| (1 dp) | (%) (1 dp) |
| 8.8 | 9.2 |
| 9.1 | 5.3 |
| 10.4 | 100.0 |
| 11.6 | 30.5 |
| 12.5 | 11.3 |
| 14.5 | 49.3 |
| 15.0 | 8.2 |
| 16.3 | 5.9 |
| 16.7 | 23.5 |
| 17.1 | 38.3 |
| 17.5 | 43.5 |
| 18.5 | 17.0 |
| 19.3 | 8.5 |
| 19.6 | 7.5 |
| 20.9 | 53.4 |
| 21.5 | 29.3 |
| 22.4 | 8.1 |
| 23.2 | 13.1 |
| 23.7 | 13.5 |
| 24.3 | 25.2 |
| 24.8 | 5.4 |
| 25.9 | 5.4 |
| 26.6 | 25.0 |
| 27.0 | 30.1 |
| 27.4 | 27.3 |
| 31.6 | 9.2 |
| 32.0 | 10.6 |
| 34.1 | 5.6 |

### FT-Raman Data

FIG. 2 shows the FT-Raman spectrum of PF-07265028 free base (Form 2), collected according to General Method 2. A full list of FT-Raman peaks (cm⁻¹) and qualitative intensities is provided in Table 3 in cm⁻¹±3 cm⁻¹.

TABLE 3
FT Raman Peak List for PF-07265028 free base Form 2 (cm⁻¹)

| Wavenumber | |
|---|---|
| cm⁻¹ ± 3 cm⁻¹ | Relative intensity |
| 422 | 3.0 |
| 467 | 4.6 |
| 499 | 3.7 |
| 561 | 4.4 |
| 633 | 3.7 |
| 738 | 7.2 |
| 774 | 3.5 |
| 801 | 4.7 |
| 826 | 11.3 |
| 888 | 9.9 |
| 938 | 6.1 |
| 974 | 20.4 |
| 991 | 27.1 |
| 1018 | 10.1 |
| 1093 | 3.5 |
| 1193 | 4.4 |
| 1212 | 4.8 |
| 1237 | 6.7 |
| 1257 | 6.8 |
| 1276 | 8.2 |
| 1306 | 5.4 |
| 1350 | 30.4 |
| 1358 | 40.6 |
| 1376 | 29.0 |
| 1402 | 19.2 |
| 1421 | 26.1 |
| 1441 | 17.1 |
| 1462 | 14.2 |
| 1503 | 100.0 |
| 1530 | 6.6 |
| 1578 | 33.0 |
| 1589 | 34.0 |
| 1700 | 40.6 |
| 2827 | 6.6 |
| 2873 | 6.1 |
| 2923 | 12.3 |
| 2949 | 9.7 |
| 2966 | 10.3 |
| 2996 | 13.2 |
| 3078 | 5.4 |

### SsNMR Data

FIG. 3 shows the carbon CP MAS spectrum of PF-07265028 free base (Form 2), which was collected according to General Method 3 (* indicates spinning sidebands). Chemical shifts are expressed in parts per million (ppm). A list of ssNMR ¹³C chemical shifts (ppm) for Form 2 is provided in Table 4 in ppm±0.2 ppm.

TABLE 4
ssNMR ¹³C Chemical Shifts for PF-07265028 free base Form 2 (ppm)

| 13C Chemical Shifts | Relative Intensity |
|---|---|
| δ ppm ± 0.2 ppm | (%) |
| 10.8 | 56.2 |
| 20.0 | 100.0 |
| 21.8 | 53.6 |
| 24.5 | 43.7 |
| 30.6 | 50.2 |
| 32.5 | 43.2 |
| 35.4 | 40.9 |
| 47.5 | 36.3 |
| 50.4 | 36.6 |
| 54.2 | 85.9 |
| 62.3 | 46.8 |
| 94.7 | 39.6 |
| 112.9 | 33.5 |
| 116.1 | 34.5 |
| 119.8 | 31.7 |
| 139.9 | 31.2 |
| 143.2 | 23.0 |
| 145.8 | 25.7 |
| 148.5 | 16.2 |
| 152.6 | 24.0 |
| 155.5 | 21.9 |
| 159.5 | 31.0 |
| 162.7 | 31.2 |
| 168.1 | 20.4 |

### Example 5

### Characterization of PF-07265028 Form 1

This example provides characterization data on PF-07265028 Form 1 from a sample obtained using a procedure disclosed in International Patent Publication No. WO2021/220185, using powder x-ray diffraction and dynamic vapor sorption to support the preparation of a disclosure. Form 1 is a crystalline hygroscopic tetrahydrate with multiple powder patterns depending on temperature and humidity. It was observed that Form 1 dehydrates at very low temperature (˜34° C.) and it does not rehydrate at high humidity conditions. In addition, Form 1 can lose crystallinity after dehydration (PXRD peak shifting and disorder observed after the water sorption experiment). The dehydration risk can pose challenges to API isolation, storage and drug product manufacture. Maintaining the proper water content is considered to be difficult for Form 1. Properties of PF-07265028 Form 1 are given in Table 5.

TABLE 5
Properties of PF-07265028 Form 1

| Analysis | Form 1 |
|---|---|
| Thermal Analysis | |
| Dehydration (TGA)- | Tₒₙₛₑₜ = ~34° C. |
| Hydrated/Solvated | ~14% weight loss |
| Hygroscopicity | |
| Moisture | Completely dehydrated at 3% RH; did not |
| Sorption/Desorption- | rehydrate at or below 10% RH |
| Hydrate (DVS) | Change in mass-ref (%) at 60% RH: ~−6.2%; |
| | 75% RH: ~−2.4% and 90% RH: ~−0.1% |
| | Post-DVS PXRD result: no change in pattern, but |
| | peak shifting and disorder observed |

Some of the powder patterns of Form 1 observed under ambient laboratory conditions are shown below.

### PXRD Data

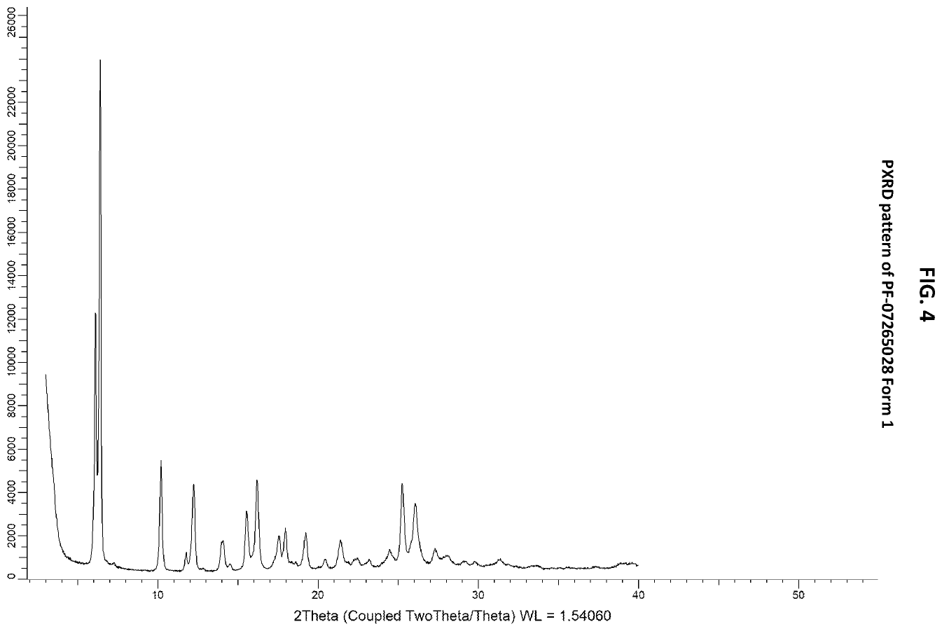

The powder x-ray diffraction (PXRD) pattern of PF-07265028 Form 1 was generated using a Bruker A25 D8 Advance diffractometer equipped with a copper (Cu) radiation source, nickel filter, and theta-2theta goniometer. The tube voltage and amperage were set to 40 kV and 40 mA, respectively, and the motorized divergence slits were set at constant illumination of 0.6 mm. Diffracted x-ray radiation was detected using a LYNXEYE energy dispersive x-ray detector, with the position sensitive detector (PSD) opening set at 3.30°. A sample was placed in a silicon low background small divot holder and smoothed with a glass slide. The powder pattern was collected at room temperature using Cu Kα1 (λ=1.5406 Å) radiation from 3.0° to 40.0° (2θ) with a step size of 0.020°, time per step of 0.3 s, and 0 rpm. Data were imported and analyzed in DIFFRAC.EVA V5.0 software. The PXRD pattern of PF-07265028 Form 1 is provided in FIG. 4.

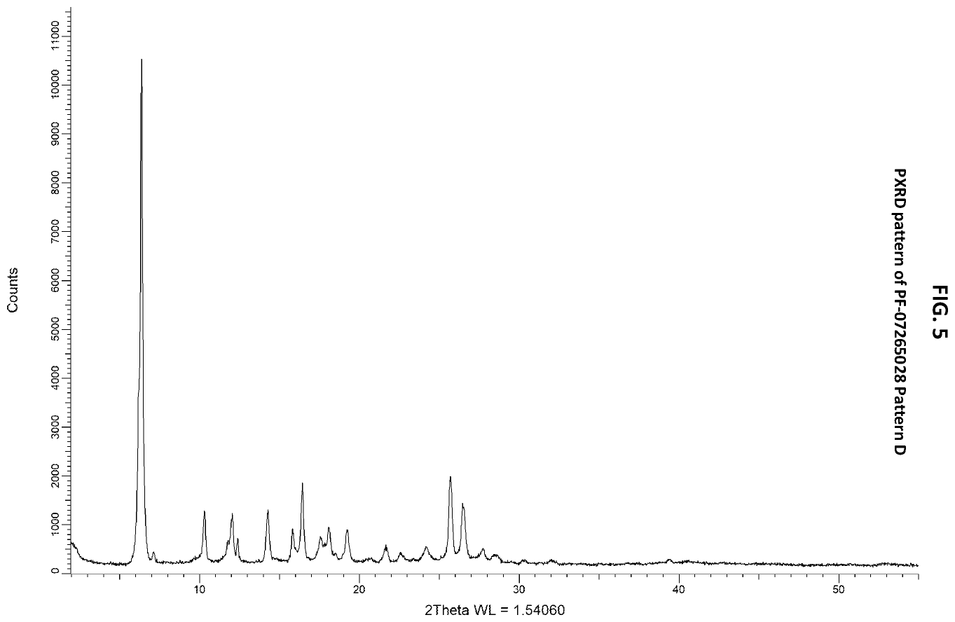

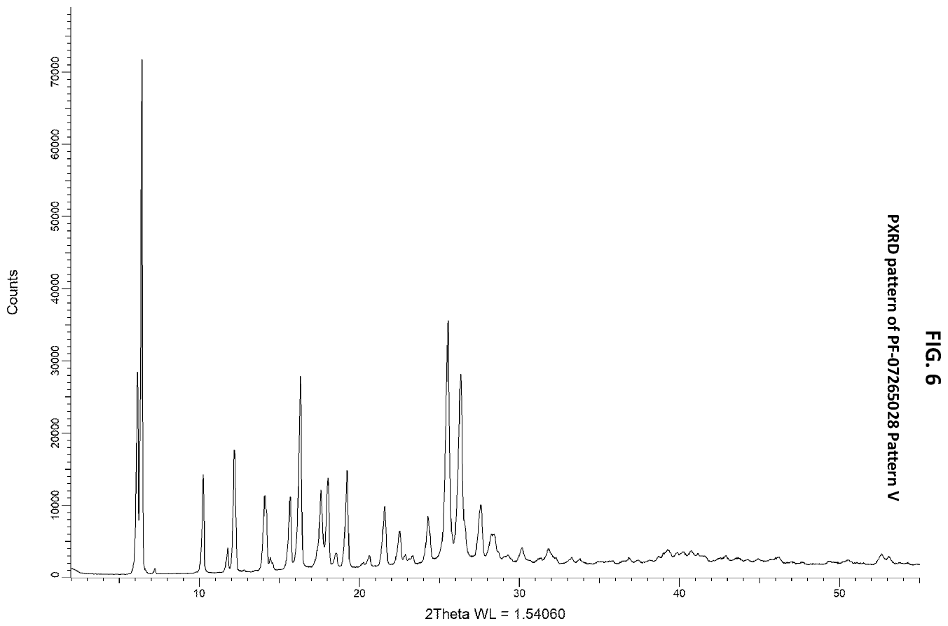

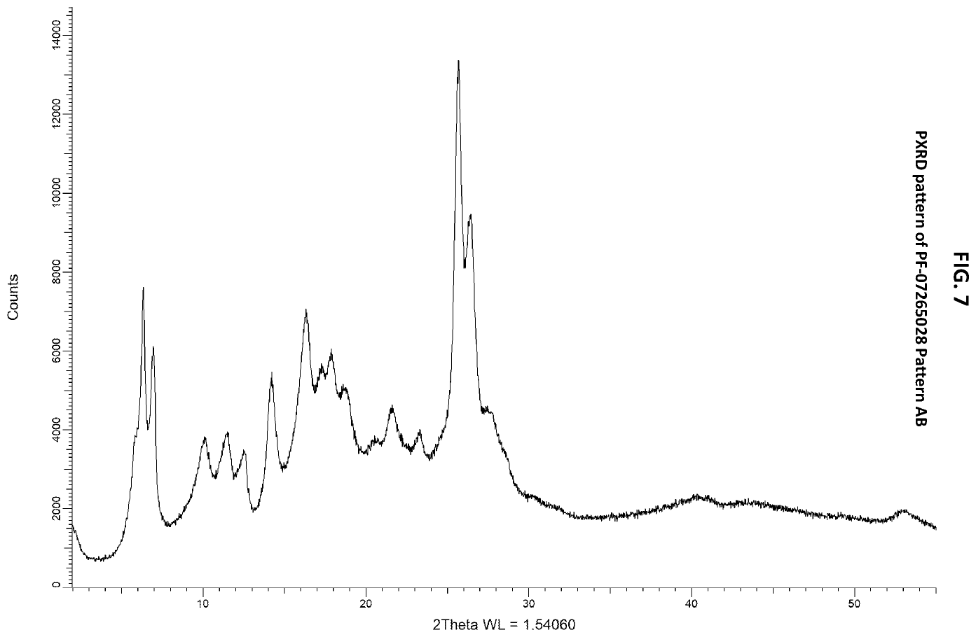

The PXRD patterns of PF-07265028 Pattern D, Pattern V, and Pattern AB were generated using a Bruker AXS D8 Endeavor diffractometer equipped with a copper (Cu) radiation source and theta-2theta goniometer. The tube voltage and amperage were set to 40 kV and 40 mA, respectively, and the motorized divergence slits were set at constant illumination of 11 mm. Diffracted x-ray radiation was detected using a LYNXEYE XE-T energy dispersive x-ray detector, with the position sensitive detector (PSD) opening set at 4.00°. A sample of PF-07265028 Pattern D was placed in a silicon low background small divot holder and smoothed with a glass slide. Powder patterns were collected immediately at room temperature (Pattern V) and then after 1 day (Pattern D) and 21 days (Pattern AB) using Cu Kα1 (λ=1.5406 Å) radiation from 2.0° to 55.0° (2θ) with a step size of 0.019°, time per step of 0.2 s, and 15 rpm. The wafer was left on the instrument for the duration of the experiment. Data were imported and analyzed in DIFFRAC.EVA V5.0 software. The PXRD patterns of PF-07265028 Pattern V, Pattern D, and Pattern AB are provided in FIG. 5, FIG. 6, and FIG. 7 respectively.

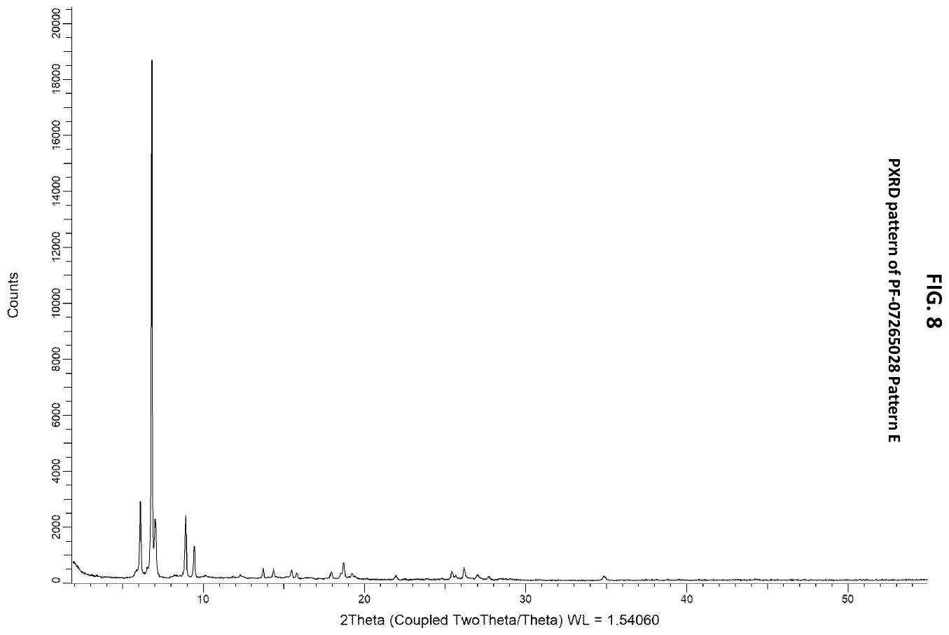

The PXRD pattern of PF-07265028 Pattern E was generated using a Bruker AXS D8 Endeavor diffractometer equipped with a copper (Cu) radiation source and theta-2theta goniometer. The tube voltage and amperage were set to 40 kV and 40 mA, respectively, and the motorized divergence slits were set at constant illumination of 11 mm. Diffracted x-ray radiation was detected using a LYNXEYE XE-T energy dispersive x-ray detector, with the position sensitive detector (PSD) opening set at 4.00°. A sample of PF-07265028 Pattern C (obtained using water:MeOH A=0.8 starting from Form 2) was isolated on filter paper, placed on a silicon low background flat plate, and smoothed with a glass slide. The powder pattern was collected at room temperature after 2 days using Cu Kα1 (λ=1.5406 Å) radiation from 2.0° to 55.0° (2θ) with a step size of 0.019°, time per step of 0.1 s, and 15 rpm. The wafer was left on the instrument for the duration of the experiment. Data were imported and analyzed in DIFFRAC.EVA V5.0 software. The PXRD pattern of PF-07265028 Pattern E is provided in FIG. 8.

Modifications may be made to the foregoing without departing from the basic aspects of the disclosure. Although the disclosure has been described in substantial detail with reference to one or more specific embodiments, those of ordinary skill in the art will recognize that changes may be made to the embodiments specifically disclosed in this application, and yet these modifications and improvements are within the scope and spirit of the disclosure.

1. A crystalline form of 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one (PF-07265028) free base (Form 2), having a powder X-ray diffraction (PXRD) pattern measured using a copper radiation source comprising peaks at 2θ values of: 9.1, 11.6, and 18.5°±0.2°.

2. The crystalline form of claim 1, having a PXRD pattern further comprising a peak at the 2θ value of: 16.7°±0.2°.

3. The crystalline form of any one of claim 1 or 2, having a Raman spectrum comprising one or more wavenumber (cm⁻¹) values selected from the group consisting of: 1700, 1402, and 2827 cm⁻¹±3 cm⁻¹.

4. The crystalline form of any one of claims 1 to 3, having a ¹³C solid state NMR spectrum comprising one or more resonance (ppm) values selected from the group consisting of: 159.5, 145.8 and 152.6 ppm±0.2 ppm relative to an external standard of L-alanine, setting its upfield resonance to 177.8 ppm.

5. A crystalline form of PF-07265028 free base (Form 2), having a ¹³C solid state NMR spectrum comprising resonance (ppm) values of: 159.5, 145.8 and 152.6 ppm±0.2 ppm, relative to an external standard of L-alanine, setting its upfield resonance to 177.8 ppm.

6. The crystalline form of claim 5, having a ¹³C solid state NMR spectrum further comprising resonance (ppm) values of 119.8 ppm±0.2 ppm.

7. The crystalline form of any one of claim 5 or 6, having a ¹³C solid state NMR spectrum further comprising resonance (ppm) values of 168.1 ppm±0.2 ppm.

8. The crystalline form of any one of claims 5 to 7, having a Raman spectrum comprising one or more wavenumber (cm⁻¹) values selected from the group consisting of: 1700, 1402, and 2827 cm⁻¹±3 cm⁻¹.

9. A crystalline form of PF-07265028 free base (Form 2), having a Raman spectrum comprising wavenumber (cm⁻¹) values of: 1700, 1402, and 2827 cm⁻¹±3 cm⁻¹.

10. The crystalline form of claim 9, having a Raman spectrum further comprising a wavenumber value of 1441 cm⁻¹±3 cm⁻¹.

11. The crystalline form of any one of claim 9 or 10, having a Raman spectrum further comprising a wavenumber value of 2996 cm⁻¹±3 cm⁻¹.

12. A crystalline form of PF-07265028 free base (Form 2), having: (a) a powder X-ray diffraction (PXRD) pattern measured using a copper radiation source comprising peaks at 2θ values of: 9.1, 11.6, and 18.5°±0.2°; (b) a Raman spectrum comprising one or more wavenumber (cm⁻¹) values selected from the group consisting of: 1700, 1402, and 2827 cm⁻¹±3 cm⁻¹; or (c) a ¹³C solid state NMR spectrum comprising one or more resonance (ppm) values selected from the group consisting of: 159.5, 145.8 and 152.6 ppm±0.2 ppm, relative to an external standard of L-alanine, setting its upfield resonance to 177.8 ppm; or a combination of two or more of (a), (b), and (c).

13. The crystalline form of any one of claims 1 to 12 which is substantially pure.

14. The crystalline form of any one of claims 1 to 13, wherein the crystalline form is anhydrous.

15. A pharmaceutical composition comprising the crystalline form of PF-07265028 free base according to any one of claims 1 to 14, and a pharmaceutically acceptable carrier or excipient.

16. A method of treating abnormal cell growth in a mammal comprising administering to the mammal a therapeutically effective amount of the crystalline form of PF-07265028 free base according to any one of claims 1 to 14, or a pharmaceutical composition according to claim 15.

17. The method of claim 16, wherein the abnormal cell growth is cancer.

18. The method of claim 17, wherein the cancer is selected from the group consisting of brain cancer, head and neck cancer, prostate cancer, bladder cancer, lung cancer, breast cancer, ovarian cancer, bone cancer, colorectal cancer, kidney cancer, liver cancer, pancreatic cancer, esophageal cancer, gastric cancer, gastroesophageal junction cancer, thyroid cancer, cervical cancer, uterine cancer, and renal cancer.

19. The method of any one of claims 16 to 18, wherein anti-tumor immunity is limited by HPK1.

20. Use of a crystalline form of PF-07265028 free base according to any one of claims 1 to 14 in a method of treating abnormal cell growth in a mammal.

21. The use of claim 20, wherein the abnormal cell growth is cancer.

22. The crystalline form of PF-07265028 free base according to any one of claims 1 to 14 for use in a method of treating abnormal cell growth in a mammal.

23. The crystalline form for use of claim 22, wherein the abnormal cell growth is cancer.

24. A method of preparing a crystalline form of 4-[(1R)-1-aminopropyl]-2-{6-[(5S)-5-methyl-6,7-dihydro-5H-pyrrolo[2,1-c][1,2,4]triazol-3-yl]pyridin-2-yl}-6-[(2R)-2-methylpyrrolidin-1-yl]-2,3-dihydro-1H-pyrrolo[3,4-c]pyridin-1-one (PF-07265028) free base (Form 2) according to any one of claims 1-14, the method comprising steps of:
providing PF-07265028 free base in a first solvent to form a first solvent solution;
adding the first solvent solution to a second solvent at a temperature from about 73° C. to about 90° C. to form a first-second-solvent solution;
removing proportion of the solvent from the first-second-solvent solution while maintaining a ratio (v/v) of the first solvent to the second solvent in the range from about 20:80 to about 2:98 to obtain a slurry; and
cooling the slurry to obtain the crystalline form of PF-07265028 free base (Form 2).

25. The method of claim 24, wherein the first solvent is selected from the group consisting of dichloromethane, ethyl ether, acetone, and mixtures thereof.

26. The method of claim 24 or 25, wherein the second solvent is selected from the group consisting of isopropyl acetate n-propyl acetate, isobutyl acetate, t-butyl acetate, and mixtures thereof.
